(19) United States
(12) Patent Application Publication
(10) Pub. No.: US 2020/0085774 A1
(43) Pub. Date: Mar. 19, 2020

(54) Omega-3 Fatty Acid Composition for Preventing and/or Treating Cachexia

(71) Applicant: BASF AS, Ludwigshafen (DE)

(72) Inventors: Hilde Nyheim, Oslo (NO); Ida Marie Wold, Oslo (NO); Laila Norrheim Larsen, Oslo (NO); Svein Olaf Hustvedt, Oslo (NO)

(21) Appl. No.: 16/470,594

(22) PCT Filed: Dec. 22, 2017
(86) PCT No.: PCT/EP2017/084422

§ 371 Date: Jun. 18, 2019

(30) Foreign Application Priority Data
Dec. 23, 2016 (NO) 20162056

(51) Int. Cl. A61K 31/202; A61K 9/00; A61K 9/48; A61K 31/232; A23L 33/12

(52) U.S. Cl. CPC A61K 31/202; A61K 9/0053; A23V 2002/00; A61K 31/232; A23L 33/12; A61K 9/4825

(57) ABSTRACT

Compositions of a polyunsaturated omega-3 fatty acid such as (5Z,8Z,11Z,14Z,17Z)-eicosa-5,8,11,14,17-pentaenoic acid (EPA) and (4Z,7Z,10Z,13Z,16Z,19Z)-docosa-4,7,10,13,16,19-hexaenoic acid (DHA) for preventing and/or treating cachexia in a subject are disclosed. The compositions contain a certain amount of the fatty acids as monoacylglycerides. Further, a method of preventing and/or treating cachexia in a subject is disclosed, including administering a composition of EPA and DHA to the subject, wherein at least a part of the fatty acids are provided in monoacylglyceride form.

### FIELD OF THE INVENTION

The present invention relates to a method of preventing and/or treating cachexia in a subject, including administering a composition of polyunsaturated omega-3 fatty acids such as of (5Z,8Z,11Z,14Z,17Z)-eicosa-5,8,11,14,17-pentaenoic acid (EPA) and (4Z,7Z,10Z,13Z,16Z,19Z)-docosa-4,7,10,13,16,19-hexaenoic acid (DHA) to the subject.

Further, the present invention discloses compositions of polyunsaturated omega-3 fatty acids such as of EPA and DHA acids for therapeutic and/or prophylactic treatment of cachexia administering the composition to the subject.

### BACKGROUND OF THE INVENTION

Dietary polyunsaturated fatty acids (PUFAs), including omega-3 PUFAs, have effects on diverse physiological processes impacting normal health and chronic diseases, such as the regulation of plasma lipid levels, cardiovascular and immune functions, insulin action, neuronal development, and visual function.

Omega-3 fatty acids, e.g. (5Z,8Z,11Z,14Z,17Z)-eicosa-5,8,11,14,17-pentaenoic acid (EPA) and (4Z,7Z,10Z,13Z,16Z,19Z)-docosa-4,7,10,13,16,19-hexaenoic acid (DHA) regulate plasma lipid levels, cardiovascular and immune functions, insulin action, neuronal development and visual function. Omega-3 fatty acids have been shown to have beneficial effects on the risk factors for cardiovascular diseases, for example hypertension and hypertriglyceridemia (HTG).

Cachexia is a syndrome or condition which may be described as a mufti-factorial syndrome defined by an ongoing loss of skeletal muscle mass and/or fat mass that cannot be reversed by conventional nutritional support and which leads to progressive functional impairment. It is secondary to an underlying disease. The pathophysiology is characterized by a negative protein and energy balance driven by a variable combination of reduced food intake and abnormal metabolism. Hence, cachexia may be defined as unintentional weight-loss and is a metabolic disorder caused by an underlying chronic disease such as cancer, chronic heart failure, HIV/AIDS, chronic obstructive pulmonary disease (COPD) or rheumatoid arthritis. Cachexia is defined as involuntary loss of at least 5% of body weight in 12 months or less in the presence of chronic illness. In addition, usually at least three of the following criteria are required: decreased muscle strength, fatigue, anorexia, low fat-free mass index, increased inflammatory markers (CRP, IL-6), anemia and low serum albumin. Loss of fat mass is a key feature of cachexia and has been attributed to increased adipocyte lipolysis.

The omega-3 fatty acids EPA and DHA have the potential to modulate multiple pathways that promote cachexia, and omega-3 fatty acid supplementation may help stabilize weight in cachexia patients on oral diets experiencing progressive, unintentional weight loss.

The use of omega-3 compounds such as EPA to treat cachexia has been suggested in the prior art. By way of example, WO 90/11073 of Cancer Research Campaign Technology Limited relates to use of EPA for treatment of cachexia, for inhibiting lipolytic activity and for inhibiting the activity of the enzyme guanidinobenzoatase. The compositions are preferably free of any other polyunsaturated fatty acids. WO 01/06983 of E. Hardman et al is directed towards formulations and methods of use of dietary supplement to prevent side effects of cancer therapy and also to reduce cachexia. The formulations comprise an oil as a concentrate prepared without an antioxidant present. US 2009/0298793 of KTB

Tumorforschungsgesellschaft MBH is directed to use of certain phospholipids, e.g. acylglycerophospholipids, including a high content of omega-3 fatty acids for treating tumor cachexia and other cancer-related problems.

The use of omega-3 fatty acids, and especially EPA, as nutrition support for cancer patients is also recommended in renowned clinical practice guidelines such as the A.S.P.E.N. (American Society for Parenteral and Enteral Nutrition) Clinical Guidelines (“Nutrition Support Therapy During Adult Anticancer Treatment and in Hematopoietic Cell Transplantation”). The guidelines recommend giving 2 g of EPA per day to help stabilize weight in cancer patients on oral diets experiencing progressive, unintentional weight loss.

Nutritional full meal drinks, some with omega-3 fatty acids in addition to inter alia carbohydrates and proteins, are the current options for nutritional management of cachexia. To obtain an effective omega-3 fatty acid dose from these drinks, the patients must drink a substantial amount, e.g. about 450 mL a day. This can be challenging, due to nausea and anorexia, and in many cases the patients are not able to comply a full dosage. The carbohydrates and proteins in the drinks do provide inter alia monosaccharides and amino acids as metabolic building blocks but have no effect on the inflammation which is one of the underlying causes of the metabolic defects in cachexia. Certain omega-3 fatty acids have a dual effect being both a nutrient and an anti-inflammatory agent.

In addition, the drinks are not suitable for patients suffering from food and nutrient allergies, which will prohibit them from taking these drinks. The result of the current situation is a lower intake of omega-3 fatty acids than what is recommended in the guidelines.

Chemotherapy, radiation and gut surgery may modify the gut function by e.g. attacking fast growing cells like epithelial cells in the outer layer of the gut wall. This can lead to reduced uptake of nutrients like omega-3 fatty acids due to reduced cell function. In addition, these patients often suffer from diarrhea which is another side effect of current cancer treatment. Thus, it is important to ensure rapid digestion and uptake of the fatty acids in the gastro intestinal tract. It is particularly important that these patients receive a formulation with optimal and enhanced bioavailability. There is hence need for new methods for treatment of cachexia patients including use of compositions which provide more easily up-take of the beneficial omega-3 fatty acids given in a delivery form which ensures high compliance and correct daily dose.

It has surprisingly been found that compositions of omega-3 fatty acids comprising a certain ratio of the fatty acids as mono-, di- and triacylglycerides enhance the digestion and uptake of the said fatty acids, in particular EPA and DHA, and the inventors have found that such compositions of polyunsaturated omega-3 fatty acids, are useful for preventing and/or treating cachexia. Such compositions may be used in the treatment or prevention of cachexia, in particular in case these are administered in an encapsulated form or provided in an enteral device.

### BRIEF SUMMARY OF THE INVENTION

In a first aspect, the present disclosure relates to a method of preventing and/or treating cachexia in a subject in need thereof, comprising administering to the subject a composition comprising a fatty acid oil mixture comprising at least 50 weight % of at least one fatty acid chosen from EPA and DHA by weight of the fatty acid oil mixture, and wherein at least 15 weight % of the fatty acids of the fatty acid oil mixture are in the form of monoacylglycerides. Particularly, it has been found that the ratio between the amount of fatty acids in form of the mono-, di- and triacylglyceride is important.

Another aspect of the disclosure relates to compositions for therapeutic and/or prophylactic treatment of cachexia wherein the composition comprises a certain amount of the fatty acids as monoacylglycerides. Hence, the invention provides a composition for therapeutic and/or prophylactic treatment of cachexia wherein the composition comprises a fatty acid oil mixture comprising at least 50 weight % of at least one fatty acid chosen from EPA and DHA by weight of the fatty acid oil mixture, and wherein at least 15 weight % of the fatty acids of the fatty acid oil mixture are in the form of monoacylglycerides. Particularly, it has been found that the ratio between the amount of fatty acids in form of the mono-, di- and triacylglyceride is important.

Likewise, the present disclosure also includes use of compositions as disclosed for therapeutic and/or prophylactic treatment of cachexia.

### BRIEF DESCRIPTION OF THE DRAWINGS

FIG. 1 provides the average, baseline corrected EPA and DHA plasma concentrations (Y-axis) versus time (X-axis) after dosing minipigs with formulations 1 or 2, wherein formulation 1 is an EPA/DHA glyceride formulation (mono-, di-, triacylglyceride) and formulation 2 is an EPA/DHA ester (ethyl ester) formulation.

FIG. 2 provides the primary oxidation products (peroxide value) in capsule formulations A and B (Y-axis) plotted versus time, in a stability chamber (X-axis), wherein capsule formulation A comprises glycerol and capsule formulation B comprises glycerol and sorbitol.

FIG. 3 provides the secondary oxidation products (anisidine value) in capsule formulations A and B (Y-axis) plotted versus time in a stability chamber (X-axis), wherein capsule formulation A comprises glycerol and capsule formulation B comprises glycerol and sorbitol.

FIG. 4 provides the average, baseline corrected EPA and DHA plasma concentrations (Y-axis) versus time (X-axis) after dosing minipigs with compositions I, H, IH or IV wherein the mono:di:tri acylglyceride ratio in the compositions are varied.

### DETAILED DESCRIPTION OF THE INVENTION

It should be noted that embodiments and features described in the context of one aspect of the present disclosure also apply to the other aspects of the invention. Particularly, the embodiments applying to the composition or formulation for preventing and/or treating cachexia according to the present disclosure also apply to the method of preventing and/or treating cachexia and likewise to the aspect of use of a compound or formulation for preventing and/or treating cachexia, all according to the present disclosure.

Particular aspects of the disclosure are described in greater detail below. The terms and definitions as used in the present application and as clarified herein are intended to represent the meaning within the present disclosure.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass up to ±10%, preferably up to ±5%, in particular up to ±2%, especially up to ±1% of a specified amount, frequency, or value.

The terms “treat,” “treating,” and “treatment” include any therapeutic application that can benefit a human or non-human mammal. Treatment may be responsive to an existing condition or it may be prophylactic, i.e., preventative. Further, the treatment can be made either acutely or chronically, and preferably chronically. By chronical treatment it is meant a treatment which continues for weeks or years. In particular the term “therapeutic treatment” includes a treatment which is responsive to an existing condition, whereas the term “prophylactic treatment” includes a treatment which is preventive with regards to a specific condition. The terms “prophylactic treatment” and “preventive treatment” are interchangeable. The terms “preventing and/or treating” and “therapeutic and/or prophylactic treatment of” may interchangeably be used. Typically, the compositions disclosed herein will be used for treating cachexia, e.g. in particular in a therapeutic treatment. However, it is also foreseen that in some cases the composition or formulations will be used for preventing or for prophylactic treatment of cachexia, for example wherein the patient has an underlying condition that may result in cachexia. Further, the “treatment” includes the use of the compositions or formulations disclosed as pharmaceuticals, medical food, food for specific medical purposes, supplements and nutraceuticals, such as in dietary or nutritional management of a cachexia patient.

The terms “administer”, “administration,” and “administering” as used herein refer to (1) providing, giving, dosing and/or prescribing by either a health practitioner or his authorized agent or under his direction a composition or formulation according to the present disclosure, and (2) putting into, taking or consuming by the human patient or person himself or herself, or non-human mammal a composition or formulation according to the present disclosure

The term “pharmaceutically effective amount” means an amount sufficient to achieve the desired pharmacological and/or therapeutic effects, i.e. an amount of the disclosed composition or formulation that is effective for its intended purpose. While individual subject/patient needs may vary, the determination of optimal ranges for effective amounts of the disclosed composition or formulation is within the skill of the art. Generally, the dosage regimen for treating a disease and/or condition with the compositions presently disclosed may be determined according to a variety of factors such as the type, age, weight, sex, diet, and/or medical condition of the subject/patient.

The term “pharmaceutical composition”/“pharmaceutical formulation” means a composition/formulation according to the present disclosure in any form suitable for medical use.

The term “mainly” means more than 50%, preferably more than 70% and most preferably more than 80% or even more than 90%.

The term “fatty acid” means a carboxylic acid with a long aliphatic chain, which is either saturated or unsaturated; a subgroup thereof are “long chain fatty acids” having at least 13 carbons and being saturated, mono-, di- or polyunsaturated; in particular “polyunsaturated fatty acids (PUFA)” are fatty acids with at least 18 carbons, preferably 18 to 22 carbons, and 3 to 6 carbon-carbon double bonds; “omega-3 fatty acids” are polyunsaturated fatty acid with at least 18 carbons, preferably 18 to 22 carbons, and 3 to 6 carbon-carbon double bonds, wherein at least one double bond is in the omega-3 position; preferred omega-3-fatty acids are EPA and DHA; “omega-6 fatty acids” are polyunsaturated fatty acid with at least 18 carbons, preferably 18 to 22 carbons, and 3 to 6 carbon-carbon double bonds, wherein at least one double bond is in the omega-6 position. The fatty acids, in particular the omega-3 fatty acids, or in another embodiment the omega-6 fatty acids, originate from natural sources, e.g. from marine sources, and is preferably from marine animals or from algae.

The terms “fatty acid”, “long chain fatty acid”, “polyunsaturated fatty acid”, “omega-3 fatty acid”, “omega-6 fatty acid” encompasses not only the free acid form of the respective fatty acid, but also derivatives, like esters, amides and salts thereof, unless expressly stated otherwise. Examples for esters are alkyl esters, like C₁-C₆-alkyl esters—e.g. methyl or ethyl esters —, like esters of polyalcohols, e.g. glycerol—for example mono-, di- and/or triacylglycerides; examples for amides are alkyl amides, like C₁-C₆ mono or di-alkyl amides etc.; example for salts are alkali salts, earth alkali salts, ammonium salts etc.; or phospholipids.

The compounds of the composition of, particularly EPA and DHA, may exist in various stereoisomeric forms, including enantiomers, diastereomers, or mixtures thereof. It will be understood that the invention encompasses all optical isomers of the compounds. Hence, compounds of the compositions that exist as diastereomers, racemates, and/or enantiomers are within the scope of the present disclosure.

In body wasting, e.g. as a result of chemotherapy, radiation or gut surgery, and cachexia, gut wall function often is modified, which may after the absorption of administered nutrients, in particular of orally administered nutrients. Changes in the gut wall are often associated with weight loss regardless of the underlying chronic disease. Cancer patients in specific, undergo rough treatments such as chemo and radiation therapy which attack fast growing cells. Epithelial cells in the outer layer of the gut wall, are especially vulnerable. The reduced cell function can lead to reduced uptake of nutrients like omega-3 fatty acids. Another side effect of the current cancer treatment is diarrhea and nausea. In addition, the gut flora in cancer patients treated in such a manner is altered which affects specific metabolite availability and absorption, and in turn affects tumor growth and cachexia. Due to all these side effects, it is therefore particularly important that cachexia patients receive a composition or formulation of omega-3 fatty acids which ensures rapid digestion and uptake in the gastro intestinal tract to achieve optimal and enhanced bioavailability of the omega-3 fatty acids. Many concentrated fish oil based products on the market today comprise omega-3 fatty acids on triacylglyceride form, i.e. three respective fatty acids are linked to a glycerol molecule, or on ethyl ester form, i.e. fatty acid compounds that lack the glyceride molecular backbone connecting adjacent fatty acids, in particular on ethyl ester form. When respective ethyl esters or triacylglycerides are digested in the gut, lipases hydrolyze the bond between the glycerol backbone/ethyl moiety and the fatty acids. The lipids need to be emulsified into the hydrophilic fluid in the stomach for lipases to access this bond and release free fatty acids that can be transported over the intestinal membrane. Bile salts are emulsifying agents secreted by the gallbladder. During the digestive period, intestinal phase signals stimulate the release of bile into the small intestine. Fatty acids in the lumen of the duodenum stimulate endocrine cells to release the hormone cholecystokinin (CCK). CCK stimulates contractions in the smooth muscle of the gallbladder. Monoacylglycerides have been shown to have self-emulsifying properties due to the hydrophilic and hydrophobic nature of the molecule.

Hence, for this patient group having a particular need for beneficial omega-3 fatty acids that are well-absorbed by the GI-tract and are easily taken up, the invention provides compositions as well as formulations of omega-3 fatty acids for cachexia patients comprising a certain proportion of fatty acids on monoacylglyceride form. It is in particular found that when mixing a certain portion of omega-3 fatty acids as monoacylglycerides with di- and triacylglycerides of omega-3 fatty acids to achieve a special ratio thereof, the composition at least partly self-emulsifies when released in the hydrophilic environment in the stomach. This eases the access of the enzymes to the bond between the glycerol backbone and the respective fatty acids and therefore enhances the digestion and uptake. In the method and composition as well as formulation for use according to the invention, it is essential that the EPA and DHA proportion is high in the fatty acid oil mixture of the composition. Thus, the invention provides a method of preventing and/or treating cachexia in a subject in need thereof, comprising administering to the subject a composition or formulation comprising a fatty acid oil mixture comprising at least 50 weight % of at least one fatty acid chosen from EPA and DHA, by weight of the fatty acid oil mixture, and wherein at least 15 weight % of the fatty acids in the fatty acid oil mixture are in the form of monoacylglycerides. The monoacylglyceride proportion should be at least 15%, such as 15-95%, more preferably at least 20%, such as 20-70%, or such as about 20-60%, or such as 20-30%, or such as 20-25% or such as 40-60%, by weight of the fatty acid oil mixture weight. Comparison studies in mini pigs, as provided in example 1 have shown improved uptake, both faster and higher, of a composition containing an omega-3 fatty acid mixture comprising more than 15% monoacylglycerides compared to a composition of omega-3 fatty acids on ethyl ester form.

Other forms of the fatty acids which may be useful to include in the monoacylglyceride (MAG)-enriched composition, formulation and for use according to the invention are free acids, salts, esters of any type; such as ethyl esters, amides, di-, or triacylglycerides and phospholipids. In one embodiment, the composition comprises mainly EPA and DHA fatty acids as glycerides, and preferably as a mixture of mono-, di- and triacylglycerides (MAG, DAG and TAG fatty acids). Bioavailability of EPA+DHA from re-esterified triacylglycerides is believed to be better than the bioavailability from ethyl esters. Hence, in one embodiment, the composition (for use) is substantially free of EPA and DHA on the alkyl ester form, such as being free of EPA- and DHA-ethyl esters. In another embodiment, the composition comprises a low amount of fatty acid alkyl esters, such as up to 10 weight %, such as 0-5 weight %.

In one embodiment, the composition comprises (beneath the monoacylglyceride component) a diacylglyceride component constituting 20-60%, such as 20-40% or such as 25-35% or such as about 30% by weight of the fatty acid content of the fatty acid oil mixture; in another embodiment In one embodiment, the composition comprises (beneath the monoacylglyceride component) a diacylglyceride component constituting 40-60%, such as 45-55, or such as about 50%, or such as about 53%, by weight of the fatty acid content of the fatty acid oil mixture.

In another embodiment, the composition comprises (beneath the monoacylglyceride component) a triacylglyceride component constituting about 0-30%, such as 10-30% by weight of the fatty acid content of the omega-3 fatty acid oil mixture.

In one embodiment the weight ratio between the mono-, di- and triacylglyceride components of the fatty acids in the fatty acid oil mixture is 15-60:25-60:0-45 or such as 15-60:40-60:0-45. In another embodiment the weight ratio between the mono-, di- and triacylglyceride component of the fatty acids in the fatty acid oil mixture is 10-30:40-60:10-30, in particular 15-25:45-60:15-35, preferably 18-25:50-58:20-30. In another embodiment the weight ratio between the mono-, di- and triacylglyceride component of the fatty acids in the fatty acid oil mixture is 30-60:20-40:10:30, in particular 45-60:25-35:10-20. Specifically exemplified compositions comprise a fatty acid oil mixture wherein the fatty acids are in form of mono-, di- and triacylglycerides in a weight ratio of about 25:50:20, 27:53:20, 60:27:10, 20:53:26, 21:53:26, or 53:30:14. In a particularly preferred embodiment, the composition comprises a fatty acid oil mixture comprising at least 50 weight %, preferably at least 60%, in particular at least 70% of at least one fatty acid chosen from EPA and DHA, by weight of the fatty acid oil mixture, and wherein the weight ratio between the mono-, di- and triacylglyceride component of the fatty acids is about 15-25:45-60:15-35. In another particularly preferred embodiment, the composition comprises a fatty acid oil mixture comprising at least 50 weight %, preferably at least 60%, in particular at least 70% of at least one fatty acid chosen from EPA and DHA, by weight of the fatty acid oil mixture, and wherein the weight ratio between the mono-, di- and triacylglyceride component of the fatty acids is about 20-30:45-55:15-30. In another preferred embodiment, the composition comprises a fatty acid oil mixture comprising at least 50 weight %, preferably at least 60%, in particular at least 70% of at least one fatty acid chosen from EPA and DHA, by weight of the fatty acid oil mixture, and wherein the weight ratio between the mono-, di- and triacyglyceride component of the fatty acids is about 50-60:25-35:10-20.

Hence, in one embodiment, the invention provides a composition, a formulation for use in therapeutic and/or prophylactic treatment of cachexia wherein the composition comprises a fatty acid oil mixture, wherein the fatty acid oil mixture comprises fatty acids in the form of mono-, di-, and triacylglycerides in a weight ratio of 15-60:25-60:0-45, respectively, wherein the fatty acid oil mixture comprises at least 50 weight % of at least one fatty acid chosen from EPA and DHA by weight of the fatty acid oil mixture.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein at least 15 weight % of the fatty acids in the fatty acid oil mixture are in the form of monoacylglycerides.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein the fatty acid oil mixture comprises the fatty acids in a mixture of mono-, di- and triacylglyceride form in a weight ratio of 15-60:25-60:0-45.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein the fatty acid oil mixture comprises the fatty acids in a mixture of mono-, di- and triacylglyceride form in a weight ratio of 20-30:45-55:15-30.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein the fatty acid oil mixture comprises the fatty acids in a mixture of mono-, di- and triacylglyceride form in a weight ratio of 50-60:25-35:10-20.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein at least 15 weight % of the fatty acids in the fatty acid oil mixture are in the form of monoacylglycerides.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein the fatty acid oil mixture comprises the fatty acids in a mixture of mono-, di- and triacylglyceride form in a weight ratio of 15-60:25-60:0-45.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein the fatty acid oil mixture comprises the fatty acids in a mixture of mono-, di- and triacylglyceride form in a weight ratio of 20-30:45-55:15-30.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein the fatty acid oil mixture comprises the fatty acids in a mixture of mono-, di- and triacylglyceride form in a weight ratio of 50-60:25-35:10-20.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, wherein the fatty acid oil mixture comprises at least 80 weight % of polyunsaturated fatty acids by weight of the fatty acid oil mixture, and wherein at least 15 weight % of the fatty acids in the fatty acid oil mixture are in the form of monoacylglycerides.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture wherein the fatty acid oil mixture comprises at least 80 weight % of polyunsaturated fatty acids by weight of the fatty acid oil mixture, and wherein the fatty acid oil mixture comprises the fatty acids in a mixture of mono-, di- and triacylglyceride form in a weight ratio of 15-60:25-60:0-45.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture at least 80 weight % of polyunsaturated fatty acids by weight of the fatty acid oil mixture, and wherein the fatty acid oil mixture comprises the fatty acids in a mixture of mono-, di- and triacylglyceride form in a weight ratio of 20-30:45-55:15-30.

In another embodiment, the invention provides a composition, a formulation for use in therapeutic and/or prophylactic treatment of cachexia, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, wherein the fatty acid oil mixture comprises at least 80 weight % of polyunsaturated fatty acids by weight of the fatty acid oil mixture, and wherein the fatty acid oil mixture comprises the fatty acids in a mixture of mono-, di- and triacylglyceride form in a weight ratio of 50-60:25-35:10-20.

In another embodiment, the MAG-enriched composition comprises minimum 85 weight % monoacylglycerides, such as more than 90 weight % monoacylglycerides, by weight of the fatty acid oil mixture, e.g. the composition comprises a fatty acid oil mixture of mono-, di- and triacylglycerides in the weight ratio of about 85-95:1-10: 0-5, or such as 93:1:0. In one specific embodiment of this, the EPA to DHA weight ratio is about 1.3:1.0. As shown in the examples such high weight ratios of the MAG-component is seen as beneficial compared to formulations of fatty acids mainly on pure ester or triacylglyceride form due to higher uptake.

The compositions according to the present invention provide for higher bioavailability of the valuable omega-3 fatty acids, in particular EPA and DHA, than when the same fatty acids are provided e.g. on ester form. As shown in Example 1, the bioavailability of the fatty acids, such as of EPA and/or DHA, is at least 20% higher than compared to a similar omega-3 fatty acid ethyl ester composition, such as e.g. 30% higher, such as 35% higher, or even 38% higher, when provided in a composition as disclosed. To contribute to a high uptake of the beneficial polyunsaturated omega-3 fatty acids, the composition, formulations and their use comprise a high concentration of these omega-3 fatty acids. Hence, the composition comprises a fatty acid oil mixture of at least 50 weight % DHA and EPA, independent of the form (mono-, and optionally di-, and optionally triacylglyceride form). More preferably, the fatty acid oil mixture comprises at least 60 weight %, e.g. at least 65 weight %, such as at least 70 weight %, such as at least 75 weight % EPA and DHA. In one embodiment, the fatty acid oil mixture comprises at least 80 weight % EPA and DHA, such as at least 83 weight % EPA and DHA, such as at least 85 weight % EPA and DHA. Further, a high concentration of the selected omega-3 fatty acids is important in order to ensure efficacy and to increase patient compliance. In the high concentrated oils, the amount of unwanted compounds such as saturated fatty acids, cholesterol, oxidation products and environmental pollutants is minimized. Such components may worsen rather than treat the condition. Several clinical studies have shown that bioaccumulation of persistent organic pollutants (POPs) may have potential adverse effects on human health and are positively associated with cancer risk.

EPA is the most widely studied omega-3 fatty acid in cachexia patients; however, it is reason to believe that also DHA, which has been present in many of the studied products, has a substantial effect. There are for example both in vitro and animal studies and one clinical trial, suggesting that a combination of DHA with anti-cancer agents often improves efficacy of anti-cancer drugs and also reduces therapy associated side effects. DHA should therefore be included in the composition for use.

In some embodiments of the present invention, the weight ratio of EPA:DHA of the fatty acid oil mixture ranges from about 1:10 to about 10:1, from about 1:8 to about 8:1, from about 1:6 to about 6:1, from about 1:5 to about 5:1, from about 1:4 to about 4:1, from about 1:3 to about 3:1, or from about 1:2 to about 2:1. In at least one embodiment, the weight ratio of EPA:DHA of the fatty acid oil mixture ranges from about 1:1 to about 2:1. In another embodiment the weight ratio of EPA:DHA of the fatty acid oil mixture ranges from about 1:1 to about 7:2, in particular from about 2:1 to 7:2, preferably from 2:1 to 3:1. Preferably, there is more EPA than DHA present in the oil mixture. In at least one embodiment, the weight ratio of EPA:DHA of the fatty acid oil mixture ranges from about 1:1 to about 4:1, such as from about 1:1 to about 3:1 or from about 1:1 to 2:1, with a weight ratio of EPA:DHA of about 2:1 being particularly preferred. Further non-limiting examples of useful weight ratios between EPA and DHA are about 1.2:1, 1.5:1, 2.5:1, 3.33:1, 1:2.5. In one embodiment, compositions with EPA and DHA in a weight ratio of about 2.3:1.0 is excluded.

In one embodiment, the fatty acid oil mixture comprises high concentrations of either EPA or DHA. In at least one embodiment, the fatty acid oil mixture comprises at least 75 percent EPA and DHA by weight of the fatty acid oil mixture, of which at least 95 percent is EPA. In another embodiment, the fatty acid oil mixture comprises at least 75 percent EPA and DHA by weight of the fatty acid oil mixture, of which at least 95 percent is DHA.

The fatty acid oil mixture of the present disclosure may comprise at least one fatty acid other than EPA and DHA, preferably another omega-3 fatty acid. Examples of such fatty acids include, but are not limited to, α-linolenic acid, heneicosapentaenoic acid, docosapentaenoic acid, eicosatetraenoic acid and octadecatetraenoic acid, and combinations thereof.

The content of omega-6 fatty acids in the fatty acid oil mixture of the composition or formulation is preferably low, e.g. lower than 10 weight % by weight of the fatty acid oil mixture, preferably lower than 8 weight %, in particular between 1 and 7 weight %. In an embodiment of the present invention the weight ratio between omega-3 fatty acids and omega-6 fatty acids are high. Preferably, the fatty acid oil mixture of the composition or formulation comprises omega-3 fatty acids and omega-6 fatty acids in a weight ratio of at least 14:1, preferably at least 16:1, in particular at least 20:1. In another embodiment, no omega-6 fatty acids are present.

In a preferred embodiment of the invention, the composition, formulation as well as the use thereof comprises a particularly low content of the unsaturated C16-4 omega-3 fatty acid hexadeca-4,7,10,13-tetraenoic acid, e.g. lower than 2 weight %, preferably lower than 1 weight % by weight of the fatty acid oil mixture. It has been shown in an in vitro and in vivo study on mice that this fatty acid of medium chain length may have chemotherapy negating effects.

In a preferred embodiment the composition, formulation for use in cachexia treatment according to the invention does not comprise another pharmaceutically active compound, e.g. like pharmaceuticals having anti-cancer activity.

In a further aspect, the invention provides a new composition, i.e. a composition as described in the above aspect. Hence, the invention provides a composition comprising a fatty acid oil mixture comprising at least 50 weight % of at least one fatty acid chosen from EPA and DHA, by weight of the fatty acid oil mixture, and wherein at least 15 weight % of the fatty acids are in the form of monoacylglycerides. The monoacylglyceride proportion should be such as 15-95%, more preferably at least 20%, such as 20-70%, or such as about 20-60%, or such as 20-30%, or such as 20-25% or such as 40-60%, by weight of the fatty acid oil mixture weight.

Other forms of the fatty acids which may be useful to include in the monoacylglyceride (MAG)-enriched composition/formulation and for use according to the invention are free acids, salts, esters of any type; such as ethyl esters, amides, di-, or triacylglycerides and phospholipids. In one embodiment, the composition comprises mainly EPA and DHA fatty acids as glycerides, and preferably as a mixture of mono-, di- and triacylglycerides (MAG, DAG and TAG fatty acids). Bioavailability of EPA+DHA from re-esterified glycerides is believed to be better than the bioavailability from ethyl esters. Hence, in one embodiment, the composition is substantially free of EPA and DHA on the alkyl ester form, such as being free of EPA- and DHA-ethyl esters. In another embodiment, the composition comprises a low amount of fatty acid alkyl esters, such as up to 10 weight %, such as 0-5 weight %.

In one embodiment, the composition comprises (beneath the monoacylglyceride component) a diacylglyceride component constituting 20-60%, such as 20-40% or such as 25-35% or such as about 30% by weight of the fatty acid content of the fatty acid oil mixture; In one embodiment, the composition comprises (beneath the monoacylglyceride component) a diacylglyceride component constituting 40-60%, such as 45-55%, or such as about 50%, or such as about 53%, by weight of the fatty acid content of the fatty acid oil mixture.

In another embodiment, the composition comprises (beneath the monoacylglyceride component) a triacylglyceride component constituting about 0-30%, such as 10-30% by weight of the fatty acid content of the omega-3 fatty acid oil mixture.

In one embodiment, the weight ratio between the mono-, di- and triacylglyceride component of the fatty acids of the fatty acid oil mixture is 15-60:25-60:0-45 or such as 15-60:40-60:0-45. In another embodiment the weight ratio between the mono-, di- and triacylglyceride component of the fatty acids in the fatty acid oil mixture is 10-30:40-60:10-30, in particular 15-25:45-60:15-35, preferably 18-25:50-58:20-30. In another embodiment the weight ratio between the mono-, di- and triacylglyceride component of the fatty acids in the fatty acid oil mixture is 30-60:20-40:10:30, in particular 45-60:25-35:10-20. Specifically exemplified compositions comprise a fatty acid oil mixture wherein the fatty acids are in form of mono-, di- and triacylglycerides in the ratio of about 25:50:20, 27:53:20, 60:27:10, 20:53:26, 21:53:26 or 53:30:14. In a particularly preferred embodiment, the composition comprises a fatty acid oil mixture comprising at least 50 weight %, preferably at least 60%, in particular at least 70% of at least one fatty acid chosen from EPA and DHA, by weight of the fatty acid oil mixture, and wherein the weight ratio between the mono-, di- and triacylglyceride component of the fatty acids is about 15-25:45-60:15-35. In another particularly preferred embodiment, the composition comprises a fatty acid oil mixture comprising at least 50 weight %, preferably at least 60%, in particular at least 70% of at least one fatty acid chosen from EPA and DHA, by weight of the fatty acid oil mixture, and wherein the weight ratio between the mono-, di- and triacylglyceride component of the fatty acids is about 20-30:45-55:15-30. In another preferred embodiment, the composition comprises a fatty acid oil mixture comprising at least 50 weight %, preferably at least 60%, in particular at least 70% of at least one fatty acid chosen from EPA and DHA, by weight of the fatty acid oil mixture, and wherein the weight ratio between the mono-, di- and triacyglyceride component of the fatty acids is about 50-60:25-35:10-20.

In another embodiment, the invention provides a composition, wherein the composition comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein at least 15 weight % of the fatty acids in the fatty acid oil mixture are in the form of monoacylglycerides.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein the fatty acid oil mixture comprises the fatty acids in a mixture of mono-, di- and triacylglyceride form in a weight ratio of 15-60:25-60:0-45.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein the fatty acid oil mixture comprises the fatty acids in a mixture of mono-, di- and triacylglyceride form in a weight ratio of 20-30:45-55:15-30.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, and wherein the fatty acid oil mixture comprises the fatty acids in a mixture of mono-, di- and triacylglyceride form in a weight ratio of 50-60:25-35:10-20.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein at least 15 weight % of the fatty acids in the fatty acid oil mixture are in the form of monoacylglycerides.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein the fatty acid oil mixture comprises the fatty acids in a mixture of mono-, di- and triacylglyceride form in a weight ratio of 15-60:25-60:0-45.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein the fatty acid oil mixture comprises the fatty acids in a mixture of mono-, di- and triacylglyceride form in a weight ratio of 20-30:45-55:15-30.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 80 weight %, preferably 85%, in particular 90% of polyunsaturated fatty acids by weight of the fatty acid oil mixture, and wherein the fatty acid oil mixture comprises the fatty acids in a mixture of mono-, di- and triacylglyceride form in a weight ratio of 50-60:25-35:10-20.

In another embodiment, the invention provides a composition, which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, wherein the fatty acid oil mixture comprises at least 80 weight % of polyunsaturated fatty acids by weight of the fatty acid oil mixture, and wherein at least 15 weight % of the fatty acids in the fatty acid oil mixture are in the form of monoacylglycerides.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture wherein the fatty acid oil mixture comprises at least 80 weight % of polyunsaturated fatty acids by weight of the fatty acid oil mixture, and wherein the fatty acid oil mixture comprises the fatty acids in a mixture of mono-, di- and triacylglyceride form in a weight ratio of 15-60:25-60:0-45.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture at least 80 weight % of polyunsaturated fatty acids by weight of the fatty acid oil mixture, and wherein the fatty acid oil mixture comprises the fatty acids in a mixture of mono-, di- and triacylglyceride form in a weight ratio of 20-30:45-55:15-30.

In another embodiment, the invention provides a composition which comprises a fatty acid oil mixture, wherein the fatty acid oil mixture comprises at least 50 weight % of at least one fatty acid chosen from EPA and DHA, wherein the fatty acid oil mixture comprises at least 65 weight %, preferably 70%, in particular 75% of omega-3 fatty acids by weight of the fatty acid oil mixture, wherein the fatty acid oil mixture comprises at least 80 weight % of polyunsaturated fatty acids by weight of the fatty acid oil mixture, and wherein the fatty acid oil mixture comprises the fatty acids in a mixture of mono-, di- and triacylglyceride form in a weight ratio of 50-60:25-35:10-20.

In another embodiment, the MAG-enriched composition comprises minimum 85 weight % monoacylglycerides, such as more than 90 weight % monoacylglycerides, by weight of the fatty acid oil mixture, e.g. the composition comprises a fatty acid oil mixture of mono-, di- and triacylglycerides in the weight ratio of about 85-95:1-10: 0-5, or such as 93:1:0. In one specific embodiment of this, the EPA to DHA weight ratio is about 1.3:1.0.

In another embodiment, the composition comprises a fatty acid oil mixture of at least 60 weight %, such as at least 65 weight %, such as at least 70 weight %, such as at least 75 weight % DHA and EPA, independent of the form. In another embodiment, the fatty acid oil mixture comprises at least 80 weight % EPA and DHA, such as at least 83 weight % EPA and DHA, such as at least 85 weight % EPA and DHA.

In another embodiment of the present invention, the weight ratio of EPA:DHA of the fatty acid oil mixture of the composition ranges from about 1:10 to about 10:1, from about 1:8 to about 8:1, from about 1:6 to about 6:1, from about 1:5 to about 5:1, from about 1:4 to about 4:1, from about 1:3 to about 3:1, or from about 1:2 to about 2:1. In at least one embodiment, the weight ratio of EPA:DHA of the fatty acid oil mixture of the composition ranges from about 1:2 to about 2:1. In another embodiment the weight ratio of EPA:DHA of the fatty acid oil mixture of the composition ranges from about 1:1 to about 7:2, in particular from about 2:1 to 7:2, preferably from 2:1 to 3:1. Preferably, there is more EPA than DHA present in the oil mixture. In at least one embodiment, the weight ratio of EPA:DHA of the fatty acid oil mixture in the composition ranges from about 1:1 to about 4:1, such as from about 1:1 to about 3:1 or from about 1:1 to 2:1, with a weight ratio of EPA:DHA of about 2:1 being particularly preferred. Further non-limiting examples of useful weight ratios between EPA and DHA in the fatty acid oil mixture of the composition are about 1.2:1, 1.5:1, 2.5:1, 3.33:1, 1:2.5. In one embodiment, compositions with EPA and DHA in a weight ratio of about 2.3:1.0 is excluded.

In one embodiment, the fatty acid oil mixture of the composition comprises high concentrations of either EPA or DHA. In at least one embodiment, the fatty acid oil mixture of the composition comprises at least 75 percent EPA and DHA by weight of the fatty acid oil mixture, of which at least 95 percent is EPA. In another embodiment, the fatty acid oil mixture of the composition comprises at least 75 percent EPA and DHA by weight of the fatty acid oil mixture, of which at least 95 percent is DHA.

The fatty acid oil mixture of the composition may comprise at least one fatty acid other than EPA and DHA, preferably another omega-3 fatty acid. Examples of such fatty acids include, but are not limited to, α-linolenic acid, heneicosapentaenoic acid, docosapentaenoic acid, eicosatetraenoic acid and octadecatetraenoic acid, and combinations thereof.

The content of omega-6 fatty acids in the fatty acid oil mixture of the composition is preferably low, e.g. lower than 10 weight % by weight of the fatty acid oil mixture, preferably lower than 8 weight %, in particular between 1 and 7 weight %. In an embodiment of the present invention the weight ratio between omega-3 fatty acids and omega-6 fatty acids are high. Preferably, the fatty acid oil mixture of the composition comprises omega-3 fatty acids and omega-6 fatty acids in a weight ratio of at least 14:1, preferably at least 16:1, in particular at least 20:1. In another embodiment, no omega-6 fatty acids are present.

In a preferred embodiment of the invention, the composition comprises a particularly low content of the unsaturated C16-4 omega-3 fatty acid hexadeca-4,7,10,13-tetraenoic acid, e.g. lower than 2 weight %, preferably lower than 1 weight % by weight of the fatty acid oil mixture.

In a preferred embodiment, the composition according to the invention does not comprise another pharmaceutically active compound, e.g. like pharmaceuticals having anti-cancer activity.

Omega-3 fatty acids in the monoacylglyceride form provide increased bioavailability compared to respective omega-3 fatty acids in other forms. Systemic bioavailability of the EPA and DHA fatty acids is increased when administered in the monoacylglyceride form, compared to as when given in other commercially available forms. These monoacylglycerides have better solubility and pharmacokinetics in physiological solution than omega-3 esters, like alkyl esters, and are more stable than the free omega-3 fatty acids. Furthermore, the particular mixture of mono-, di- and triacylglycerides is believed to behave like a self-emulsifying system, enhancing the surface area of the oil droplets in the stomach, allowing for better access for the lipases which digest the oil further. The EPA and DHA are hence more quickly absorbed. An omega-3 fatty acid composition as disclosed, preferably comprising a high concentration of EPA and DHA in an optimized mixture of EPA and DHA as mono-, di-, and triacylglycerides is believed to meet the requirements to bioavailability, solubility and stability.

Further, the compositions according to the present invention can be produced in industrial scale in a cost-efficient way, keeping the production costs at a competitive level, comparable to the production costs of compositions comprising high concentrations of EPA and/or DHA on the ethyl ester or triacylglyceride form.

An increased uptake of the PUFAs can be measured e.g. as an increase in absolute amounts of EPA and DHA in fasting serum triglycerides, cholesterol esters and phospholipids.

In the method of treating cachexia, or in the use of the composition according to the invention, the composition may for example at least one of;
inhibit lipolysis in adipose tissue e.g. the compounds of the composition act as inhibitors of the lipases that are upregulated in cachectic adipose tissue and, thus reduce the loss of fat mass.
reduce an abnormal, e.g. elevated level of cyclic adenylic acid (cAMP) produced in adipocytes;
reduce the concentration of triglycerides in serum by upregulating the expression/activity of Lipoprotein lipase (LPL). Increased levels of leukemia inhibitory factor (LIF) and tumor necrosis factor-α (TNFα) are registered in cachetic patients. LIF and TNFα have been found to decrease both the mRNA expression and activity of LPL in cultured adipocytes.
have an anti-inflammatory effect; specific eicosanoids, prostaglandins and leukotrienes derived from arachidonic acid (omega-6) precursors feature pro-inflammatory properties. Omega-3 fatty acid supplementation competes in the same metabolic pathway that leads to the synthesis of arachidonic acid and its precursors and therefore reduces the levels of omega-3 pro-inflammatory agents. In addition, EPA-derived mediators, like PGE3 and LTB₅. are often much less biologically active than those produced from arachidonic acid. EPA and DHA also give rise to anti-inflammatory and inflammation resolving resolvins and related compounds (e.g., protectins) through pathways involving cyclooxygenase and lipoxygenase enzymes. Resolvin D1 inhibits IL-13 production, and protectin D1 inhibits TNF and IL-13 production. Inflammatory cytokines such as IL-1, IL-6 and TNFα are increased and play a significant role in the pathogenesis of cancer cachexia. Inflammation is a central driver of muscle wasting in the neoplastic state, therefore attenuation of inflammatory mediators by decreasing the production by n-6 fatty acid derivatives may improve muscle mass in cancer cachexia.
Inhibit the proteolysis inducing factor (PH); PH is a cachetic factor produced by cancer cells linked to the signaling system initiating protein degradation.
Inhibit ubiquitin-proteosome induced muscle proteolysis.

Cachexia is a secondary condition associated with a chronic disease. In addition to treating the cachexia, treatment of the underlying condition is important. Hence, in one embodiment the method of the invention includes a treatment of cachexia as disclosed combined with treatment of the underlying disease. For example, in some embodiments, the underlying disease is one or more of cancer, chronic heart failure, HIV/AIDS, chronic obstructive pulmonary disease (COPD), or rheumatoid arthritis. In one preferred embodiment of the invention, the underlying disease is cancer, and the composition is primarily for treating tumor-induced cachexia. Cachexia is most common in patients having lung, gastro, pancreatic and head and neck cancer. In one embodiment, the composition is particularly for treatment of cachexia patients diagnosed with either one of lung, gastro, pancreatic and head and neck cancer. The combined treatment may for instance include steps to ensure that the composition for use according to the invention is administered in a time and dosing program coordinated with the treatment performed to treat the underlying condition. In an preferred embodiment of the invention the combined treatment of cachexia patients is achieved by separately administering the composition or formulation of the present and the pharmaceutical against the underlaying disease. For cancer patients, such treatment of the underlying condition may include for instance administration of chemotherapy intended to kill or reduce the growth and spread of cancer cells, and radiation therapy including beam radiation and use of radioactive pellets or solutions. Further, in one embodiment of the invention the method or composition for use may also positively affect the underlying condition or the treatment of this. Particularly DHA is shown, both in vitro, in animal studies, and a clinical study, to improve efficacy of anticancer drugs and also reduce therapy-associated side-effects. Incorporation of DHA in cellular membranes improves drug uptake, whereas increased lipid peroxidation is another mechanism for DHA-mediated enhanced efficacy of anticancer drugs. For instance, in addition to treat cachexia, using the composition as disclosed comprising EPA and DHA fatty acids as glycerides, and particularly MAGs, may reduce the side effects of therapeutics used to treat the underlying condition, such as cancer. Hence, the invention provides a method as disclosed further reducing the side effects of chemotherapy or radiation therapy. As there also are strong indications that DHA and EPA selectively increase the sensitivity of tumor tissue but not non-tumor tissue to chemotherapy, the method of treating cachexia as suggested may also augment the effect of chemotherapy. Accordingly, the invention provides a method as disclosed further increasing the efficacy of chemotherapy or radiation therapy of cancer. The treatment or use according to the invention may in turn result in either of a reduction of tumor growth or metastatic spread, a reduction of side effects, promote survival and an improvement of the cachectic situation. Hence, the present disclosure further encompasses methods and use of the composition as disclosed, for improving at least one parameter associated with cachexia such as to alleviate or correct either of: loss in body mass, loss of muscle strength, weakness/fatigue, loss of appetite, myosteatosis, or to improve quality of life and/or reduce days spent in hospital.

Cancer patients with low muscle mass have poorer performance status, lower quality of life, shorter survival, are more likely to experience chemotherapy toxicity, and are at higher risk of becoming bedridden and having longer hospital stays, compared with patients with muscle mass above a certain cut point. In cancer patients, low muscle mass and myosteatosis, a pathological deposition of fat into skeletal muscle, appears to occur concurrently with low plasma levels of EPA and DHA. Several clinical studies have reported an improvement in muscle mass when fish oil—which is rich in omega-3 fatty acids—is provided to cancer patients. Furthermore, as listed above, the effects of proteolysis inducing factor (PIF) are also inhibited by EPA. PIF binds to certain surface receptors on the skeletal muscle, which are linked to the signaling system that initiates protein degradation in response to PH. Regarding reduction in skeletal muscle proteins in persons with cachexia, dietary omega-3 fatty acid supplementation is shown to increase the rate of muscle protein synthesis. EPA also blocks ubiquitin-proteosome induced muscle proteolysis, and thereby reduces the catabolism of muscle protein.

As described above, in addition to that the EPA/DHA MAG-including composition according to the present invention has a high bioavailability, providing an increased level of EPA and DHA in plasma, red blood cells and tissue, the EPA/DHA MAG-including composition according to the present invention, in particular the monoacylglycerides, may also increase the production of beneficial metabolites such as resolvins acting as pro resolving mediators. These may have an anti-inflammatory or resolution-stimulating activity.

The compositions presently disclosed may be formulated in variable forms, such as in oral administration forms, e.g., tablets or soft or hard gelatin capsules, chewable capsules or beads, or alternatively as a fluid composition for tube feeding. The tablet or capsule dosage form can be of any shape suitable for oral administration, such as spherical, oval, ellipsoidal, cube-shaped, regular, and/or irregular shaped. Conventional formulation techniques known in the art may be used to formulate the compounds according to the present disclosure. In a preferred embodiment, the composition for use is encapsulated, e.g. in the form of a gelatin capsule or a tablet. In one embodiment, a formulation, in particular an oral formulation, is provided, which comprises the composition of the present invention, and a gelatin capsule, wherein the composition is encapsulated in a gelatin capsule, in particular the gelatin capsule containing additives to improve stability and/or taste or smell. In one embodiment, the capsule is flavored. In one embodiment, the gelatin capsule comprises gelatin and at least one plasticizer, e.g. at least one of glycerol and sorbitol, such as a mixture of glycerol and sorbitol. Such formulation allows an increased stability of the fatty acid oil mixture.

As the intended patient group typically may experience problems e.g. with nausea, or may have swallowing problems, the formulation according to the present application has either of an appealing taste, appealing smell, and appealing look, and improved stability. A comparison study provided in Example 5 showed that both taste and smell were improved for gelatin capsules containing both glycerol and sorbitol as plasticizers compared to capsules containing glycerol alone.

In an embodiment the oral formulation of the present invention comprise a gelatin capsule, wherein the gelatin capsule comprises gelatin and at least one plasticizer, e.g. at least one of glycerol and sorbitol, such as a mixture of glycerol and sorbitol, and which comprise 300-1000 mg fill weight of composition comprising the fatty acid oil mixture according to the present invention, preferably 500-800 mg, in particular 600-700 mg, e.g. 600 mg. As another example, a gelatin capsule may comprise 20-40 mg fill weight of the fatty acid oil mixture, given in a sachet to add up to required daily dose, further comprising a mixture of glycerol and sorbitol in the shell formulation.

In one specific embodiment, the oral formulation comprises the composition of the present invention, and a gelatin capsule, wherein the gelatin capsules is filled with the composition as described and the gelatin capsule comprises e.g. 10-25 w/w % glycerol and e.g. 5-20 w/w % sorbitol based on the weight of the capsule. The design for a specific soft gelatin capsule formulation involves appropriate selection of the shell and fill composition. This is followed by optimization of the two to allow for efficient production of a chemically and physically stable product with the desired properties. Glycerol is a commonly used plasticizer in soft gelatin capsules. Stability studies on encapsulated high concentrate omega-3 oils, as provided in Example 2, have shown that adding sorbitol as an additional plasticizer improves stability of the oil, thereby reducing the formation of oxidation products. As a result, the amount of antioxidant may potentially be reduced. In addition to improved stability of the fatty acid oil mixture, sorbitol may also improve the smell of the soft gelatin capsules and enhance the capsule finish gloss for a premium appearance. This is beneficial for the cachexia patient group, particularly as the group may experience problems e.g. with nausea, or may have swallowing problems. Of the same reasons, it is important that the capsule size is not too large. The capsule size should be a small to medium sized gelatin capsule, e.g. 300-1000 mg fill weight, preferably 500-800 mg, in particular 600-700 mg, e.g. about 600 mg. When using such small capsules, it is highly beneficial that the composition filled in the capsules has a high concentration of the beneficial fatty acids EPA and DHA. The invention provides a unique composition of beneficial fatty acids in a form that enhances the digestion and uptake of these, combined with an encapsulation that ensures high stability of the fatty acids and a premium smell, taste and appearance.

In another embodiment, the composition is formulated as a fluid composition for tube feeding to enable administration of this valuable omega-3 fatty acid composition to patients who cannot obtain nutrition by swallowing. Hence, the composition for use in treatment of cachexia is administered to a subject from a small prefilled dispenser via a tube port, as an enteral nutrition. Such tube formulation comprises the composition of the present invention, and an enteral device, wherein the composition is filled into the enteral device. Such enteral devices are described in WO 2016/120318, the description thereof is incorporated by reference herein and composition according to the present invention may be administered according to the description of WO2016/120318. Hence, the composition is prefilled in an enteral devise e.g. the enteral devise being a handheld dispenser, wherein said dispenser comprises i) a flexible body portion providing a reservoir for the composition according to the present invention, the flexible body portion comprising a wall material compatible with the composition, wherein the flexible body portion having arranged thereto; ii) an outlet connectable with a feeding tube. The composition is as disclosed above, e.g. it comprises a fatty acid oil mixture, the fatty acid oil mixture comprises fatty acids in the form of mono-, di-, and triacylglycerides in a weight ratio of 15-60:25-60:0-45, respectively, the fatty acid oil mixture comprises at least 50 weight % of at least one fatty acid chosen from EPA and DHA.

In a particular embodiment, the above-mentioned handheld dispenser is for one-time use. In another embodiment, the above-mentioned handheld dispenser has a volume of 1 to 30 mL In another embodiment, the outlet of the above-mentioned handheld dispenser is a Luer Slip part or an ENFit connector part. In another embodiment, the wall material of the handheld dispenser comprises a multibarrier layer, in particular such multibarrier layer comprises three to seven, preferably five to seven layers. In another embodiment, the above-mentioned handheld dispenser does not comprise an inlet. In another particular embodiment, at least two or more of the before mentioned embodiments may be combined.

The composition presently disclosed may comprise at least one non-active pharmaceutical ingredient, i.e., excipient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, solvents, carriers, diluents, binders, fillers, sweeteners, aromas, pH modifiers, viscosity modifiers, antioxidants, extenders, humectants, disintegrating agents, solution-retarding agents, absorption accelerators, wetting agents, absorbents, lubricants, coloring agents, dispersing agents, and preservatives. Excipients may have more than one role or function, or may be classified in more than one group; classifications are descriptive only and are not intended to be limiting.

In some embodiments, for example, the at least one excipient may be chosen from corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof. In some embodiments, the compositions presently disclosed comprise a pharmaceutically acceptable antioxidant, e.g., tocopherol such as alpha-tocopherol, beta-tocopherol, gamma-tocopherol, and delta-tocopherol, or mixtures thereof, BHA such as 2-tert-butyl-4-hydroxyanisole and 3-tert-butyl-4-hydroxyanisole, or mixtures thereof and BHT (3,5-di-tert-butyl-4-hydroxytoluene), or ascorbyl palmitate or mixtures thereof.

In an embodiment, the composition of the present invention comprises at least 60 weight % of the fatty acid oil mixture described above, by weight of the composition, in particular at least 70%, 80%, 90%, 95%, 98%, 99%. In a particular embodiment 0.01 to 1%, preferably 0.05 to 0.5 weight % of an antioxidant, by weight of the composition, is comprised in the composition. embodiment

A suitable daily dosage of the composition according to the present invention may range from about 100 mg to about 6 g. For example, in some embodiments, the daily dose of the composition ranges from about 200 mg to about 4 g, from about 250 mg to about 3 g, from about 300 mg to about 2 g, from about 400 mg to about 1000 mg. In at least one embodiment, the daily dose of EPA and DHA, from the composition as disclosed, ranges from about 200 mg to about 4 g. In at least one embodiment, the daily dose of EPA and DHA is about 200 mg, about 300 mg, about 400 mg, about 500 mg, about 600 mg, about 700 mg, about 800 mg, about 900 mg, about 1000 mg, about 1500 mg, about 2000 mg, about 3000 mg or about 4000 mg. In another embodiment, the daily dose of EPA and DHA range from 1 to 4 g, in particular 1.5 to 3.5 g, preferably from 2 to 3 g. The composition may be administered, for example, once, twice, or three times per day. In at least one embodiment, the composition is administered in an amount providing a daily dose of EPA and DHA ranging from about 500 mg to about 4000 mg, in particular 500 mg to 3000 mg.

Treatment of patients should preferably start when the patient is diagnosed for the respective chronic disease, as a preventive step. Research shows that cancer patients with blood cell membrane omega-3 fatty acid concentrations above a certain level have better prognosis and treatment effects and less cachexia and adverse effects than patients with low levels of omega-3 fatty acids therein. Therefore, the composition should be given to the patient at the time when the disease, e.g. cancer, is detected and continued during the treatment, such as the cancer treatments. For further optimizing absorption of the EPA and DHA fatty acids, the composition should preferably be taken with food. A benefit with the composition of the invention, is that this can be administered independently of other nutrition, and the diet can hence be regulated freely, e.g. taking any allergies or specific needs into consideration.

In some embodiments of the present disclosure, the composition acts as an active pharmaceutical ingredient (API). In some embodiments, the fatty acid of the composition is present in a pharmaceutically-acceptable amount, the composition is then called a pharmaceutical composition, and is for medical use. As used herein, the term “pharmaceutically-effective amount” means an amount sufficient to treat, e.g., reduce and/or alleviate the effects, symptoms, etc. In at least some embodiments, the composition does not comprise an additional active agent, e.g. an additional pharmaceutically active agent. In this embodiment, the composition may be used in pharmaceutical treatment of patients with cachexia. Where the composition is a pharmaceutical composition, the composition preferably comprises at least 70 percent of at least one of EPA and DHA by weight of the fatty acids in the composition. For example, in one embodiment, the composition comprises at least 75 percent EPA and DHA, such as at least 80 percent, at least 85 percent, at least 90 percent, or at least 95 percent, by weight of the fatty acid therein. In another embodiment, the composition according to the invention is a food supplement or a nutritional supplement. Further, in one embodiment the composition is a food supplement, a dietary supplement, a nutritional supplement, over-the-counter (OCT) supplement, medical food, or pharmaceutical grade supplement. In a related embodiment, the invention provides a composition selected from the group of Enteral Formulas for Special Medical Use, Foods for Specified Health Uses, Food for Special Medical Purposes (FSMP), Food for Special Dietary Use (FSDU), Medical Nutrition and a Medical Food. Such composition is particularly suited for patients having a deficiency of certain nutrients, such as the omega-3 fatty acids (n-3 PUFAs). Such composition is typically administered to the subject under medical supervision. In this embodiment, the treatment includes a nutritional treatment. Accordingly, the term “treatment” encompass both pharmaceutical treatment and nutritional treatment. Accordingly, the composition is for use in the treatment or dietary management of a cachexia patient. In a preferred embodiment, the composition is, or forms part of, Medical Food suitable for administration to cachexia patients. The composition and the method of the invention has the ability to correct a nutritional deficiency in a target population. A deficiency means that the patient has a level of omeag-3 fatty acids (n-3 PUFA) below the average level or that the target population has a special need. Accordingly, the patient may not have obtained sufficient levels from their diet to cover the increased need related to the existing health condition, or have a particular benefit of high levels of EPA and/or other PUFAs.

Some specific embodiments of the invention are listed below, wherein the features of an embodiment may be combined with the features of another embodiment or with features of several other embodiment.

An oral formulation for use in the nutritional management of un-intentional weight loss and/or muscle loss and/or to maintain lean body mass and/or improve quality of life and/or in a cancer patient, wherein the oral formulation comprises a composition which comprises a fatty acid oil mixture, the fatty acid oil mixture comprises fatty acids in the form of mono-, di-, and triacylglycerides in a weight ratio of 20-30:45-55:15-30 or about 50-60:25-35:10-20, respectively; the fatty acid oil mixture comprises at least 65 weight % of EPA and DHA; wherein the EPA:DHA ratio is from about 1:1 to 3:1; and the oral formulation comprises a gelatin capsule comprising at least one plasticizer, in particular glycerol and sorbitol, wherein the composition is encapsulated in the gelatin capsule; In particular such oral formulation is administered to the cancer patient in a daily dose of 2 to 4 grams EPA and DHA.

An oral formulation for use in the nutritional management of un-intentional weight loss in a human, wherein the use improves at least one parameter associated with cachexia, such as to alleviate or correct either of: loss in body mass, loss of muscle strength, weakness/fatigue, loss of appetite, myosteatosis, or to improve quality of life and/or reduce days spent in hospital for a cancer patient, wherein the oral formulation comprises a composition which comprises a fatty acid oil mixture, the fatty acid oil mixture comprises fatty acids in the form of mono-, di-, and triacylglycerides in a weight ratio of 20-30:45-55:15-30 or about 50-60:25-35:10-20, respectively; the fatty acid oil mixture comprises at least 65 weight % of EPA and DHA; wherein the EPA:DHA ratio is from about 1:1 to 3:1; and the oral formulation comprises a gelatin capsule comprising at least one plasticizer, in particular glycerol and sorbitol, and wherein the composition is encapsulated in the gelatin capsule; In particular such oral formulation is administered to the human in a daily dose of 2 to 4 grams EPA and DHA.

Use of an oral formulation, wherein the oral formulation comprises a composition which comprises a fatty acid oil mixture, the fatty acid oil mixture comprises fatty acids in the form of mono-, di-, and triacylglycerides in a weight ratio of 20-30:45-55:15-30 or about 50-60:25-35:10-20, respectively; the fatty acid oil mixture comprises at least 65 weight % of EPA and DHA; wherein the EPA:DHA ratio is from about 1:1 to 3:1; and the oral formulation comprises a gelatin capsule comprising at least one plasticizer, in particular glycerol and sorbitol, and wherein the composition is encapsulated in the gelatin capsule, in the nutritional management of un-intentional weight loss in a human, wherein the use improves at least one parameter associated with cachexia, such as to alleviate or correct: loss in body mass, loss of muscle strength, weakness/fatigue, loss of appetite, myosteatosis and to improve quality of life and/or reduce days spent in hospital for a cancer patient.

Use of an oral formulation, wherein the oral formulation comprises a composition, wherein the composition comprises a fatty acid oil mixture, the fatty acid oil mixture comprises fatty acids in the form of mono-, di-, and triacylglycerides in a weight ratio of about 25:50:25 or about 50-60:25-35:10-20, respectively; the fatty acid oil mixture comprises at least 65 weight % of EPA and DHA; wherein the EPA:DHA ratio is from about 1:1 to 3:1; and the oral formulation comprises a gelatin capsule comprising at least one plasticizer, in particular glycerol and sorbitol, and wherein the composition is encapsulated in the gelatin capsule, in the nutritional management of un-intentional weight loss in a human, wherein the use alleviate or correct either of: loss in body mass and/or loss of muscle strength.

Special Medical Use product, Foods for Specified Health Uses product, Food for Special Medical Purposes (FSMP) product, Food for Special Dietary Use (FSDU) product, Medical Nutrition product and Medical Food product consisting essentially of an oral formulation, wherein the oral formulation comprises a composition, wherein the composition comprises a fatty acid oil mixture, the fatty acid oil mixture comprises fatty acids in the form of mono-, di-, and triacylglycerides in a weight ratio of 20-30:45-55:15-30 or about 50-60:25-35:10-20, respectively; the fatty acid oil mixture comprises at least 65 weight % of EPA DHA; wherein the EPA:DHA ratio is from about 1:1 to 3:1; and the oral formulation comprises a gelatin capsule comprising at least one plasticizer, in particular glycerol and sorbitol, and wherein the composition is encapsulated in the gelatin capsule; in particular for use in the nutritional management of un-intentional weight loss in a human. Such use improves at least one parameter associated with cachexia, such as to alleviate or correct either of: loss inbody mass, loss of muscle strength, weakness/fatigue, loss of appetite, myosteatosis and to improve quality of life and/or reduce days spent in hospital, for a cancer patient.

The compositions for use according to the invention, wherein at least 15 weight %, of the EPA and DHA fatty acids are in the form of monoacylglycerides, is prepared in a transesterification process from a highly concentrated fatty acid ester oil as a starting material, e.g. from a concentrated composition of EPA- and DHA alkyl esters. In this main process step, such fatty acids esters are converted to their glyceride form by reacting the esters with glycerol, yielding the product oil, with alcohol formed as a by-product. The reaction is preferably facilitated by a catalyst, such as an enzyme. The product oil prepared typically comprises a mixture of mono-, di and triacylglycerides. Typically, parameters that may be regulated to steer the amount of mono-, di- and triacylglycerides prepared are the amount of glycerol used, the reaction time and the amount of catalyst. The conversion to glycerides is preferably performed under vacuum, by adding the fatty acid esters, such as fatty acid ethyl esters of EPA and DHA, and glycerol to the reactor already containing the catalyst. The catalyst is added to the reactor by suction with vacuum and is reused over multiple batches before being depleted and requiring change. The amount of enzyme affects reaction time, but not product quality, and is not product critical. To further up-concentrate the amount of either of the mono-, di- or triacylglycerides of EPA and DHA, to obtain an optimal composition and ratio between these, one or more distillations may typically be done.

In addition to the main process step outlined above directed to converting fatty acid esters to glycerides, and hence preparing the beneficial mixture of mono-, di-, and triacylglycerides, the process for preparing the composition may include the following steps: A crude oil, such as crude fish oil, may be subject to a stripping process such as outlined in WO2004/007654 of the applicant, such stripping process includes a thin-film evaporation process, a molecular distillation or a short-path distillation of a fatty acid oil mixture, using a volatile working fluid. The volatile working fluid may either be mixed with the fatty acid oil mixture to be purified or may be added in the stripping process separately. In this stripping process, environmental pollutants are removed from the crude oil wherein the main components are different fatty acids in the form of triacylglycerides. The volatile working fluid comprises at least one of a fatty acid ester, a fatty acid amide and a free fatty acid. Pollutants are stripped off together with the volatile working fluid.

Preferably, the stripping processing step is followed by at least a step to separate the glycerol backbone from the fatty acid chains, creating fatty acid esters. This is typically done by subjecting the stripped oil mixture to at least one trans-esterification reaction with a C1-C6 alcohol under substantially anhydrous conditions, and in the presence of a suitable catalyst (a chemical catalyst or an enzyme) to convert the fatty acids present as triacylglycerides in the oil mixture into esters of the corresponding alkyl alcohol. Thereafter, the fatty acid ester product obtained may be purified, i.e., by separation of the fatty acids and recovery of the wanted fatty acid esters, such as highly concentrated EPA- and DHA-alkyl esters. This separation may for example include distillations, preferably one or more molecular distillations, or alternatively by other methods, such as chromatographic separations. The highly concentrated fatty acids esters prepared and selected will then be used as the feed for the main step, reassembling the glyceride form, but with only EPA and DHA fatty acids.

The process for preparing the composition for use according to the invention may include further process steps, e.g. a) purification steps to remove impurities or unwanted components, b) steps to increase stability or increase concentration, and c) chemical reaction steps. Such further purifications steps may e.g. include any of alkali refining/deacidification e.g. to remove free fatty acids and water-soluble impurities, degumming, bleaching to remove oxidation products and colored components and deodorization to remove volatile components causing taste and odor. The concentration steps may include any of extractions and urea complexation, in addition to e.g. distillations and chromatography.

In a preferred embodiment, the fatty acid oil mixture of the composition for use according to the invention is prepared in a process comprising the following steps;
i) Stripping of a crude oil comprising fatty acids as triacylglycerides with a volatile working fluid to remove pollutants;
ii) Transesterification of stripped triacylglyceride fatty acids, reacting triacylglycerides with an alcohol, to prepare corresponding fatty acid esters;
iii) Transesterification of fatty acid esters to glyceride form, by reacting esters with glycerol, preparing mono-, di- and triacylglycerides of EPA and DHA.

Some specific embodiments of the invention are listed below in the example section.

In the following certain embodiments are described:
1. A composition comprising a fatty acid oil mixture comprising at least 50 weight % of at least one fatty acid chosen from EPA and DHA by weight of the fatty acid oil mixture, and wherein at least 15 weight % of the fatty acids of the fatty acid oil mixture are in the form of monoacylglycerides.
2. A composition according to embodiment 1, wherein at least 20, preferably 40-60 weight % of the fatty acids of the fatty acid oil mixture are in the form of monoacylglycerides.
3. A composition according to embodiment 1, wherein 15-95, preferably 20-70, in particular 20-60, especially 20-30, in particular preferably 20-25 weight % of the fatty acids of the fatty acid oil mixture are in the form of monoacylglycerides.
4. A composition according to anyone of embodiments 1 to 3, wherein the amount of fatty acid alkyl esters is at most up to 10, preferably between 0-5 weight %, by weight of the fatty acid oil mixture.
5. A composition according to anyone of embodiments 1 to 4 being substantially free of EPA and DHA in alkylester form.
6. A composition according to anyone of embodiments 1 to 5, wherein 20-60%, preferably 20-40%, in particular 25-35%, especially about 30 weight % of the fatty acids of the fatty acid oil mixture are in the form of diacylglycerides.
7. A composition according to anyone of embodiments 1 to 5, wherein 40-60, preferably 45-55, in particular about 50 or about 53 weight % of the fatty acids of the fatty acid oil mixture are in the form of diacylglycerides.
8. A composition according to any one of embodiments 1 to 7, wherein 0-30, preferably 10-30 weight % of the fatty acids of the fatty acid oil mixture are in the form of triacylglycerides.
9. A composition according to any one of embodiments 1 or 6, wherein weight ratio between the mono-, di- and triacylglyceride components of the fatty acids in the fatty acid oil mixture is 15-60:25-60:0-45, preferably 15-60:40-60:0-45.
10. A composition according to any one of embodiments 1 or 6, wherein weight ratio between the mono-, di- and triacylglyceride components of the fatty acids in the fatty acid oil mixture is 10-30:40-60:10-30, preferably 15-25:45-60:15-35, in particular 18-25:50-58:20-30.
11. A composition according to any one of embodiments 1 or 6, wherein weight ratio between the mono-, di- and triacylglyceride components of the fatty acids in the fatty acid oil mixture is 30-60:20-40:10:30, preferably 45-60:25-35:10-20.
12. A composition according to any one of embodiments 1 or 6, wherein weight ratio between the mono-, di- and triacylglyceride components of the fatty acids in the fatty acid oil mixture is about 25:50:20, 27:53:20, 60:27:10, 20:53:26, 21:53:26, or 53:30:14.
13. A composition according to anyone of embodiments 1 to 12, wherein the fatty acid oil mixture comprises at least 60, preferably at least 70 weight % of at least one fatty acid chosen from EPA and DHA, by weight of the fatty acid oil mixture.
14. A composition according to anyone of embodiments 1 to 13, wherein the fatty acid oil mixture comprises at least 60, preferably at least 65, in particular at least 70, especially at least 75 weight % EPA and DHA, by weight of the fatty acid oil mixture.
15. A composition according to anyone of embodiments 1 to 13, wherein the fatty acid oil mixture comprises at least 80, preferably at least 83, in particular at least 85 weight % EPA and DHA, by weight of the fatty acid oil mixture.
16. A composition according to embodiment 15, wherein weight ratio between the mono-, di- and triacylglyceride components of the fatty acids in the fatty acid oil mixture is 15-25:45-60:15-35.
17. A composition according to embodiment 15, wherein weight ratio between the mono-, di- and triacylglyceride components of the fatty acids in the fatty acid oil mixture is 20-30:45-55:15-30.
18. A composition according to embodiment 15, wherein weight ratio between the mono-, di- and triacylglyceride components of the fatty acids in the fatty acid oil mixture is 50-60:25-35:10-20.
19. A composition according to anyone of embodiments 1 to 18, wherein the fatty acid oil mixture comprises at least 65, preferably 70, in particular 75 weight % of omega-3 fatty acids by weight of the fatty acid oil mixture.
20. A composition according to anyone of embodiments 1 to 19, wherein the fatty acid oil mixture comprises at least 80, preferably 85, in particular 90 weight % of polyunsaturated fatty acids by weight of the fatty acid oil mixture.
21. A composition according to anyone of embodiments 1 or 6 which comprises minimum 85, preferably 90 weight % monoacylglycerides, by weight of the fatty acid oil mixture.
22. A composition according to embodiment 21 wherein weight ratio between the mono-, di- and triacylglyceride components of the fatty acids in the fatty acid oil mixture is 85-95:1-10: 0-5, preferably about 93:1:0.
23. A composition according to embodiment 21 wherein weight ratio of EPA:DHA is about 1.3:1.0.
24. A composition according to anyone of embodiments 1 to 22, wherein the weight ratio of EPA:DHA is from about 1:10 to about 10:1, preferably from about 1:8 to about 8:1, in particular from about 1:6 to about 6:1, especially from about 1:5 to about 5:1, in particular preferably from about 1:4 to about 4:1.
25. A composition according to embodiment 24, wherein the weight ratio of EPA:DHA is from about 1:3 to about 3:1, preferably from about 1:2 to about 2:1, in particular from about 1:1 to about 2:1.
26. A composition according to embodiment 24, wherein the weight ratio of EPA:DHA is from about 1:1 to about 7:2, preferably from about 2:1 to 7:2, in particular from 2:1 to 3:1.
27. A composition according to embodiment 24, wherein the weight ratio of EPA:DHA is from about 1:1 to about 4:1, preferably from about 1:1 to 3:1, in particular from about 1:1 to about 2:1.
28. A composition according to anyone of embodiments 1 to 22, wherein the fatty acid oil mixture comprises at least 75 weight % EPA and DHA by weight of the fatty acid oil mixture, of which at least 95 weight % is EPA.
29. A composition according to anyone of embodiments 1 to 22, wherein the fatty acid oil mixture comprises at least 75 weight % EPA and DHA by weight of the fatty acid oil mixture, of which at least 95 weight % is DHA.
30. A composition according to anyone of embodiments 1 to 29, wherein the fatty acid oil mixture comprises at least one fatty acid other than EPA and DHA, preferably another omega-3 fatty acid, in particular at least one of α-linolenic acid, heneicosapentaenoic acid, docosapentaenoic acid, eicosatetraenoic acid and octadecatetraenoic acid.
31. A composition according to anyone of embodiments 1 to 30, wherein the fatty acid oil mixture comprises at most 10, preferably at most 8, in particular from 1 to 7 weight % of omega-6 fatty acid(s) by weight of the fatty acid oil mixture.
32. A composition according to anyone of embodiments 1 to 31, wherein the weight ratio of omega-3 fatty acids to omega-6 fatty acids is at least 14:1, preferably at least 16:1, in particular at least 20:1.
33. A composition according to anyone of embodiments 1 to 30, wherein the fatty acid oil mixture comprises no omega-6 fatty acids.
34. A composition according to anyone of embodiments 1 to 33, wherein the fatty acid oil mixture comprises at most 2, preferably at most 1 weight % hexadeca-4,7,10,13-tetraenoic acid by weight of the fatty acid oil mixture.
35. A composition according to anyone of embodiments 1 to 34, wherein the composition does not comprise another pharmaceutically active compound.
36. A composition according to anyone of embodiments 1 to 35 wherein the composition comprises an antioxidant, preferably a tocopherol, such as alpha-tocopherol, beta-tocopherol, gamma-tocopherol or delta-tocopherol, or mixtures thereof; BHA, such as 2-tert-butyl-4-hydroxyanisole or 3-tert-butyl-4-hydroxyanisole, or mixtures thereof; BHT (3,5-di-tert-butyl-4-hydroxytoluene) or ascorbyl palmitate; or mixtures thereof.
37. A composition according to anyone of embodiments 1 to 36 wherein the composition comprises at least 60, preferably at least 70, in particular 80, especially 90, in particular preferably 95 weight % of the fatty acid oil mixture, by weight of the composition.
38. A composition according to embodiment 37, wherein the composition comprises at least 96, preferably at least 97, in particular 98, especially 99 weight % of the fatty acid oil mixture, by weight of the composition.
39. A composition according to embodiment 37 or 38 wherein the composition comprises 0.01 to 1, preferably 0.05 to 0.5 weight % of an antioxidant, by weight of the composition.
40. A composition according to anyone of embodiments 1 to 39, wherein the bioavailability of the fatty acids of the fatty acid oil mixture is at least 20% higher compared to a similar fatty acid ethyl ester composition.
41. A formulation comprising
   a composition according to anyone of the embodiments 1 to 40, and
   a shell,
   wherein the composition is encapsulated in the shell.
42. A formulation according to embodiment 41, wherein the formulation is an oral administration form, preferably a gel capsule, a chewable capsule, a tablet or a bead, in particular a gelatine capsule.
43. An oral formulation comprising
   a composition according to anyone of the embodiments 1 to 40, and
   a gelatin capsule,
   wherein the composition is encapsulated in the gelatin capsule, preferably in a gelatin capsule containing additives to improve stability and/or taste and smell of the formulation.
44. An oral formulation according to embodiment 43, wherein the gelatine capsule comprises at least gelatin and at least one plasticizer, preferably the at least one plasticizer is glycerol or sorbitol.
45. An oral formulation according to embodiment 44, wherein the gelatin capsule comprises 10-25 w/w % glycerol and/or 5-20 w/w % sorbitol based on the weight of the gelatin capsule.
46. An oral formulation according to embodiment 44 or 45, wherein the gelatine capsule comprises at least gelatin, glycerol and sorbitol.
47. An oral formulation according to anyone of embodiments 43 to 46, wherein gelatine capsule is filled with 300-100, preferably 500-800, in particular 600-700 mg of the composition.
48. A formulation according to embodiment 41, wherein the shell is an enteral devise.
49. A tube formulation comprising
   a composition according to anyone of the embodiments 1 to 40, and
   enteral device,
   wherein the composition is filled into the enteral device.
50. A tube formulation according to embodiment 49, wherein the enteral devise is a handheld dispenser, preferably wherein the handheld dispenser comprises i) a flexible body portion providing a reservoir for the composition according to the present invention, the flexible body portion comprising a wall material compatible with the composition, wherein the flexible body portion having arranged thereto; ii) an outlet connectable with a feeding tube.
51. Composition according to anyone of the embodiments 1 to 40 for use in the therapeutic and/or prophylactic treatment of cachexia.
52. Composition according to anyone of the embodiments 1 to 40 for use in the therapeutic and/or prophylactic treatment of cachexia of patients wherein the underlying disease of cachexia is one or more of cancer, chronic heart failure, HIV/AIDS, chronic obstructive pulmonary disease (COPD) and rheumatoid arthritis.
53. Composition according to anyone of the embodiments 1 to 40 for use in the therapeutic and/or prophylactic treatment of cachexia of patients with diagnosed cancer, preferably, with diagnosed lung, gastro, pancreatic and/or head and neck cancer.
54. Composition according to anyone of embodiments 1 to 40 for use in the therapeutic and/or prophylactic treatment of cachexia of patients, wherein the use for therapeutic and/or prophylactic treatment further reduces side effects of therapeutics used for treatment of the underlying disease of cachexia, or augments the effect of this.
55. Composition according to anyone of the embodiments 1 to 40 for use in the therapeutic and/or prophylactic treatment of cachexia, wherein at least one of the following parameter is alleviate or corrected: loss in body mass, loss of muscle strength, weakness/fatigue, loss of appetite, myosteatosis, increase in quality of life and/or reduction of days spent in hospital.
56. Composition according to anyone of embodiments 1 to 40 for use in the treatment of cachexia, wherein the treatment is a a nutritional treatment by dietary management of the cachexia.
57. Composition according to anyone of embodiments 1 to 40, for use in the treatment of cachexia, wherein the composition is selected from the group of Enteral Formulas for Special Medical Use, Foods for Specified Health Uses, Food for Special Medical Purposes (FSMP), Food for Special Dietary Use (FSDU), Medical Nutrition and Medical Food, and the use is a nutritional treatment.
58. Composition according to anyone of embodiments 1 to 40, for use in the treatment of cachexia, wherein the use is combined with treatment of the underlying disease, and wherein the composition is administered in a time and dosing program coordinated with the treatment program of the underlying disease.
59. Composition according to anyone of embodiments 1 to 40 for use in the nutritional management of un-intentional weight loss and/or muscle loss and/or to maintain lean body mass and/or improve quality of life in a cancer patient.
60. Formulation according to anyone of the embodiments 42 to 47 for use in the therapeutic and/or prophylactic treatment of cachexia.
61. Formulation according to anyone of the embodiments 42 to 47 for use in the therapeutic and/or prophylactic treatment of cachexia of patients wherein the underlying disease of cachexia is one or more of cancer, chronic heart failure, HIV/AIDS, chronic obstructive pulmonary disease (COPD) and rheumatoid arthritis.
62. Formulation according to anyone of the embodiments 42 to 47 for use in the therapeutic and/or prophylactic treatment of cachexia of patients with diagnosed cancer, preferably, with diagnosed lung, gastro, pancreatic and/or head and neck cancer.
63. Formulation according to anyone of the embodiments 42 to 47 for use in the therapeutic and/or prophylactic treatment of cachexia of patients, wherein the use for therapeutic and/or prophylactic treatment further reduces side effects of therapeutics used for treatment of the underlying disease of cachexia, or augments the effect of this.
64. Formulation according to anyone of the embodiments 42 to 47 for use in the therapeutic and/or prophylactic treatment of cachexia, wherein at least one of the following parameter is alleviate or corrected: loss in body mass, loss of muscle strength, weakness/fatigue, loss of appetite, myosteatosis, increase in quality of life and/or reduction of days spent in hospital.
65. Formulation according to anyone of embodiments 42 to 47 for use in the treatment of cachexia, wherein the treatment is a nutritional treatment by dietary management of the cachexia.
66. Formulation according to anyone of embodiments 42-47, for use in the treatment of cachexia, wherein the composition is selected from the group of Enteral Formulas for Special Medical Use, Foods for Specified Health Uses, Food for Special Medical Purposes (FSMP), Food for Special Dietary Use (FSDU), Medical Nutrition and Medical Food, and the use is a nutritional treatment.
67. Formulation according to anyone of embodiments 42-47 for use in the treatment of cachexia, wherein the use is combined with treatment of the underlying disease, and wherein the composition is administered in a time and dosing program coordinated with the treatment program of the underlying disease.
68. Formulation according to anyone of embodiments 42-47 for use in the nutritional management of un-intentional weight loss and/or muscle loss and/or to maintain lean body mass and/or improve quality of life in a cancer patient.
69. A method for preventing and/or treating cachexia in a subject in need thereof, comprising administering to a subject in need a composition according to anyone of embodiment 1 to 40 or a formulation according to anyone of embodiments 42 to 47.
70. The method of embodiment 69, wherein the method comprises administering from about 100 mg to 6 g, preferably from about 200 mg to about 4 g, in particular from about 250 mg to about 3 g, especially from about 300 mg to about 2 g, in particular preferably from about 400 mg to 1 g per day of the composition.
71. The method of embodiment 69 or 70, wherein the method comprises administering from about 200 mg to 4 g, preferably from about 1 to 4 g, in particular from about 1.5 to 3.5 g, especially from about 2 to 3 g per day EPA and DHA.
72. The method of anyone of embodiments 69 to 71, wherein at least one of the following in the treated subjects occurs: at least partial inhibition of the lipolytic activity in adipose tissue; reduction of abnormal level of cyclic adenylic acid (cAMP); at least partial inhibition of guanidinobenzoatase; reduction of circulating triacylglycerols (TAGs); regulation of LDL, VLDL, LPL, LIF or TNFα levels; causing an anti-inflammatory effect; at least partly inhibiting the proteolysis inducing factor (PH); at least partly inhibiting ubiquitin-proteosome induced muscle proteolysis, and increasing production of resolvins.
73. The method of anyone of embodiments 69 to 72, wherein the composition or formulation is food supplement, a dietary supplement, a nutritional supplement, over-the-counter (OCT) supplement, medical food, or pharmaceutical grade supplement.
74. Use of the composition according to anyone of embodiments 1 to 40 or a formulation according to anyone of embodiments 42 to 47 for preventing and/or treating cachexia in a subject in need thereof.
75. Special Medical Use product, Foods for Specified Health Uses product, Food for Special Medical Purposes (FSMP) product, Food for Special Dietary Use (FSDU) product, Medical Nutrition product and Medical Food product comprising a composition according to anyone of embodiments 1-40 or a formulation according to anyone of embodiments 42-47 for use in the nutritional management of un-intentional weight loss in a human, wherein the treatment improves at least one parameter associated with cachexia, such as to alleviate or correct at least one of: loss in body mass, loss of muscle strength, weakness/fatigue, loss of appetite, myosteatosis, or improves quality of life and/or reduce days spent in hospital for said patient.
76. The composition according to embodiment 1 for use in therapeutic and/or prophylactic treatment of cachexia, wherein the composition comprises EPA and DHA fatty acids mainly as a mixture of mono-, di- and triacylglycerides.
77. The composition according to anyone of embodiments 1 or 76 for use in therapeutic and/or prophylactic treatment of cachexia, wherein the composition comprises a diacylglyceride component constituting 40-60% by weight of the fatty acid content of the fatty acid oil mixture.
78. The composition according to anyone of embodiments 1 or 76 to 77 for use for use in therapeutic and/or prophylactic treatment of cachexia, wherein the composition comprises a triacy glyceride component constituting about 0-45% by weight of the fatty acid content of the fatty acid oil mixture.
79. The composition according to anyone of embodiments 1 or 76 to 78 for use for use in therapeutic and/or prophylactic treatment of cachexia, wherein the composition comprises mono-, di- and triacyglyceride components of EPA and DHA in a weight ratio of 15-60:40-60:0-45.
80. The composition according to anyone of embodiments 1 or 76 to 79 for use for use in therapeutic and/or prophylactic treatment of cachexia, wherein the fatty acid oil mixture of the composition comprises at least 65 weight %, such as at least 70 weight %, such as at least 75 weight % EPA and DHA.
81. The composition according to anyone of embodiments 1 or 76 to 80 for use for use in therapeutic and/or prophylactic treatment of cachexia, wherein the composition comprises a fatty acid oil mixture wherein the weight ratio of EPA:DHA ranges from about 1:10 to about 10:1.
82. The composition according to anyone of embodiments 1 or 76 to 81 for use for use in therapeutic and/or prophylactic treatment of cachexia, wherein the composition at least one of; inhibits lipolytic activity in adipose tissue; reduces an abnormal level of cyclic adenylic acid (cAMP); inhibits activity of guanidinobenzoatase; reduces circulating triacylglycerols (TAGs); regulates LDL, VLDL, LPL, LIF or TNFα levels; has an anti-inflammatory effect; inhibits the proteolysis inducing factor (PH); inhibits ubiquitin-proteosome induced muscle proteolysis or increases production of resolvins.
83. The composition according to anyone of embodiments 1 or 76 to 82 for use for use in therapeutic and/or prophylactic treatment of cachexia, wherein the underlying disease of cachexia is one or more of cancer, chronic heart failure, HIV/AIDS, chronic obstructive pulmonary disease (COPD) and rheumatoid arthritis.
84. The composition according to anyone of embodiments 1 or 76 to 83 for use in therapeutic and/or prophylactic treatment of cachexia, wherein the use further reduces side effects of therapeutics used for treatment of the underlying disease of cachexia, or augments the effect of this.
85. The composition according to anyone of embodiments 1 or 76 to 84 for use in therapeutic and/or prophylactic treatment of cachexia, wherein the use is combined with treatment of the underlying disease, wherein the composition is administered in a time and dosing program coordinated with the treatment program of the underlying disease.
86. The composition according to anyone of embodiments 1 or 76 to 85 for use in therapeutic and/or prophylactic treatment of cachexia, wherein the composition is encapsulated in a gelatin capsule containing additives to improve stability and/or taste or smell.
87. The composition according to embodiment 86 for use for use in therapeutic and/or prophylactic treatment of cachexia, wherein the encapsulated composition contains a mixture of glycerol and sorbitol.
88. The composition according to anyone of embodiments 1 or 76 to 87 for use in therapeutic and/or prophylactic treatment of cachexia, wherein said composition is administered in an amount providing a daily dose of EPA and DHA of between about 500 mg to about 3000 mg.
89. A method of preventing and/or treating cachexia in a subject in need thereof, comprising administering to the subject a composition comprising a fatty acid oil mixture comprising at least 50 weight % of at least one fatty acid chosen from EPA and DHA, by weight of the fatty acid oil mixture, and wherein at least 15 weight % of the fatty acids are in the form of monoacylglycerides.

### EXAMPLES

The present disclosure may be further described by the following non-limiting examples, in which standard techniques known to the skilled chemist and techniques analogous to those described in these examples may be used where appropriate. It is understood that the skilled artisan will envision additional embodiments consistent with the disclosure provided herein.

### Example 1: Improved Uptake of EPA/DHA as Glyceride Mixture; Minipig Study

A pharmacokinetic study in 8 Gottingen minipigs was run to compare the bioavailability of a monoacylglyceride formulation with an equivalent omega-3 fatty acid oil on ethyl ester form. The minipigs were dosed in a cross-over design to obtain data on both oils from all eight animals. OH #1 comprised 46 w/w % EPA and 38 w/w % DHA formulated as a mixture of 20 w/w % monoacylglycerides, 53 w/w % diacylglycerides and 26 w/w % triacylglycerides (glyceride mix). OH #2 comprised 46 w/w % EPA and 38 w/w % DHA on ethyl ester form. Each animal was dosed with 2 gram oil. A total of 9 blood samples were taken from the animals after dosing at the following time points: pre-treatment and 1, 2, 4, 6, 8, 12, 24 and 36 hours post-treatment. Plasma concentrations of EPA and DHA were analyzed and the results were dose correlated.

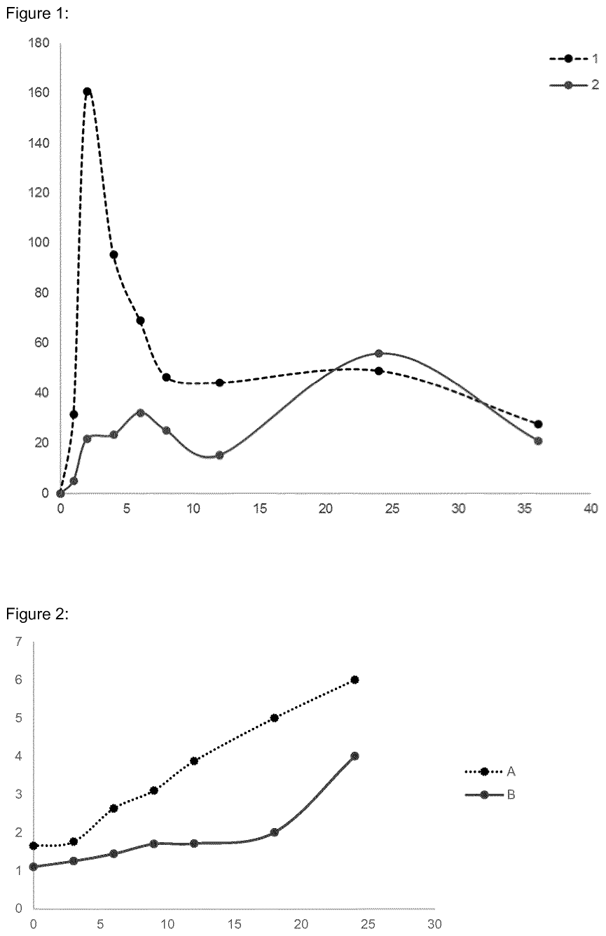

FIG. 1 provides the average, baseline corrected EPA and DHA plasma concentrations (μg/ml) versus time (hours) after dosing minipigs with the oils #1 or #2. The area under the curve provides values from zero to 36 hours after dosing (AUC(0-36)). The data is also shown in Table 1.

TABLE 1
Area under curve from start of dosing to 36 hours after dosing
AUC0-36

| | AVE oil #1 | AVE oil #2 |
|---|---|---|
| EPA | 1290 | 845 |
| DHA | 556 | 307 |
| EPA + DHA | 1846 | 1142 |

The results show a 38% higher amount of EPA and DHA in blood plasma when dosing EPA and DHA as a glyceride mix (OH #1) compared to OH #2, confirming the increased bioavailability of the fatty acids of the OH #1 (glyceride mix).

### Example 2. Capsule Shell Formulation

OH #2, as defined in Example 1, was encapsulated into soft gelatin capsules made from two different shell formulations. Capsule shell formulation A comprised gelatin and 32% w/w dry weight glycerol. Formulation B comprised gelatin and a mix of 19% w/w dry weight glycerol and 12% w/w dry sorbitol. The capsules were packed into high density polyethylene (HDPE) bottles with HDPE lids and placed in a temperature and humidity controlled stability chamber (25° C./60% RH) for 24 months. Oxidation of the oil inside the capsules were tested at various time points: start, 2, 6, 9, 12, 18 and 24 months. Oxidation was measured by detection of peroxide value (primary oxidation products) and anisidine value (secondary oxidation products).

FIG. 2 provides the primary oxidation products (peroxide value) in capsule formulations A and B (Y-axis) plotted versus time (months) in a stability chamber (X-axis).

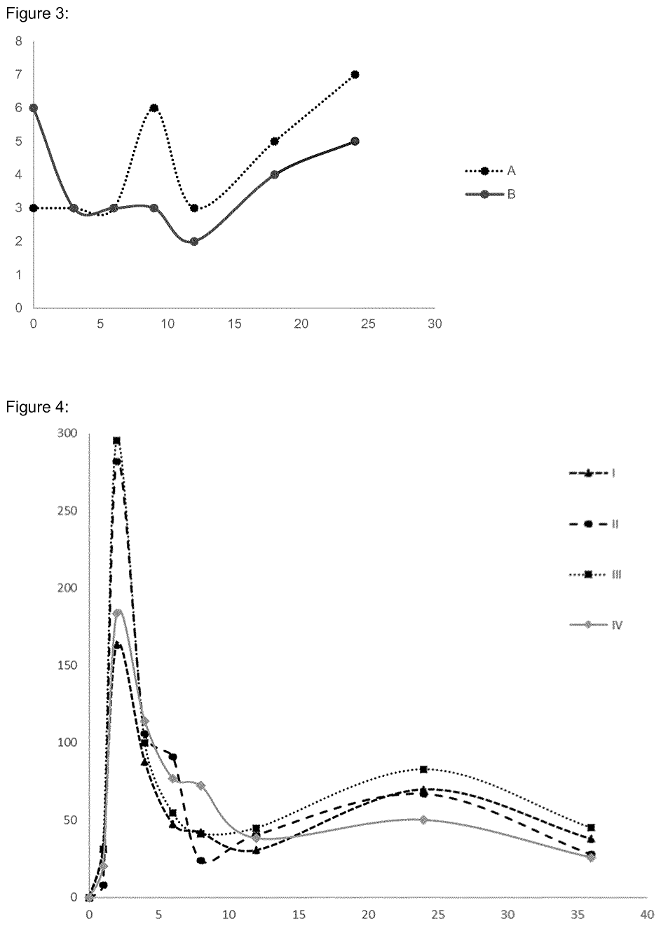

FIG. 3 provides the secondary oxidation products (anisidine value) in capsule formulations A and B (Y-axis) plotted versus time (months) in a stability chamber (X-axis).

The results provided in FIGS. 2 and 3 show that capsules with capsule shell formulation B, containing sorbitol, give lower formation of both primary and secondary oxidation products. Capsules made with shell formulation B hence provide a better protection of the oil.

### Example 3: Bioavailability Study in Minipigs to Optimize Glyceride Formulation

A pharmacokinetic study in 4×4 Gottingen minipigs was run to compare the bioavailability of four different mono:di:tri ratio acylglyceride compositions. AH the oil mixture compositions comprised 15 w/w % EPA and 50 w/w % DHA formulated as a mixture of mono-, di- and triacylglycerides.

Composition # I comprised 3 w/w % monoacylglycerides, 61 w/w % diacylglycerides and 36 w/w % triacylglycerides;

Composition # H comprised 21 w/w % monoacylglycerides, 53 w/w % diacylglycerides and 26 w/w % triacylglycerides;

Composition # IH comprised 53 w/w % monoacylglycerides, 30 w/w % diacylglycerides and 14 w/w % triacylglycerides;

Composition # IV comprised 91 w/w % monoacylglycerides, 1 w/w % diacylglycerides and 0 w/w % triacylglycerides.

Each animal was dosed with 2 gram of the respective oil mixture composition. A total of 9 blood samples were taken from the animals after dosing at the following time points: pre-treatment and 1, 2, 4, 6, 8, 12, 24 and 36 hours post-treatment. Plasma concentrations of EPA and DHA were analyzed and the results were dose correlated.

FIG. 4 provides the average, baseline corrected EPA and DHA plasma concentrations (μg/ml) versus time (hours) after dosing minipigs with Composition #I, II, III or IV. The area under the curve provides the values from zero to 36 hours after dosing (AUC(0-36)). The Composition # H (21:53:26) and Composition # IH (53:30:14) showed superior results. The in vivo data confirms in vitro lipolysis rate measurements. In Table 2 the respective data for the AUC-24 is shown.

TABLE 2
Area under curve from start of dosing to 24 hours after dosing
AUC 0-24

| | AVE oil #I | AVE oil #II | AVE oil #III | AVE oil #IV |
|---|---|---|---|---|
| EPA | 593 | 723 | 786 | 809 |
| DHA | 742 | 897 | 976 | 692 |
| EPA + DHA | 1335 | 1619 | 1762 | 1501 |

### Example 4. Stability Trial for Composition of 90% MAG in a Gelatin Capsule

1000 mg high concentrated omega-3 fatty acid oil comprising 46 w/w % EPA and 38 w/w on a ˜90 w/w % monoacylglyceride form was encapsulated into soft gelatin capsules (no sorbitol as plasticizer). The capsules were packed into high density polyethylene (HDPE) bottles with HDPE lids and placed in a temperature and humidity controlled stability chamber at accelerated (40° C./75% RH) and real time (25° C./60% RH) conditions for 6 and 12 months respectively. Capsule shell quality was measured by different parameters at initial, 1 and 3 months. The results are shown in Table 3.

The results show that the capsules, with such a high concentration of the fatty acids on the monoacylglyceride form, were outside of the desired specification criteria on hardness and water content of the shell which are two important shell quality parameters.

TABLE 3
soft gelatin capsules filled with 1000 mg omega-3 fatty oil, 90%
monoacylglyceride form, 460 mg EPA and 380 mg DHA,
accelerated conditions

| Preliminary | | | | |
|---|---|---|---|---|
| results | Specification | Initial value | 1 month | 3 months |
| Hardness of | 8-11 | 10.2 | 6.4 | 5.3 |
| capsule (N) | | | | |
| Water content | Max 0.5 | 1.4 | 1.5 | 1.5 |
| (w/%) | | | | |

### Example 5. Influence of Plasticizers in the Gelatin Capsule Improves Smell and Taste

High concentrated omega-3 fatty acid oil comprising 50 w/w % EPA and 20 w/w % DHA on triacylglyceride form, was encapsulated into soft gelatin capsules made from two different shell formulations. Capsule shell formulation A comprised gelatin and 32% w/w dry weight glycerol. Formulation B comprised gelatin and a mix of 19% w/w dry weight glycerol and 12% w/w dry sorbitol. The capsules were packed into high density polyethylene (HDPE) bottles with HDPE lids and kept for approximately 6 months at room temperature (non-controlled environment, to mimic user storage conditions). 8 independent subjects (2 males, 6 females) received one bottle of each formulation A and B, opened each bottle, and smelled and tasted the capsules. The subjects were instructed to report whether and to what extend they could smell and or taste any difference and to state which formulation they preferred. AH the participants could smell a difference between the two formulations. 75% preferred the smell of the formulation with both glycerol and sorbitol added. 87% could taste a difference between the two capsule types. 60% preferred the taste of the formulation with both glycerol and sorbitol.

1. A composition comprising a fatty acid oil mixture comprising at least 50 weight % of at least one fatty acid chosen from EPA and DHA by weight of the fatty acid oil mixture, and wherein at least 15 weight % of the fatty acids of the fatty acid oil mixture are in the form of monoacylglycerides, for use in the therapeutic and/or prophylactic treatment of cachexia.

2. A composition according to embodiment 1, wherein 15-95, preferably 20-70, in particular 20-60, especially 20-30, in particular preferably 20-25 weight % of the fatty acids of the fatty acid oil mixture are in the form of monoacylglycerides.

3. A composition according to anyone of embodiments 1 to 2, wherein the amount of fatty acid alkyl esters is at most up to 10, preferably between 0-5 weight %, by weight of the fatty acid oil mixture.

4. A composition according to anyone of embodiments 1 to 3, wherein 20-60%, preferably 20-40%, in particular 25-35%, especially about 30 weight % of the fatty acids of the fatty acid oil mixture are in the form of diacylglycerides.

5. A composition according to any one of embodiments 1 to 4, wherein 0-30, preferably 10-30 weight % of the fatty acids of the fatty acid oil mixture are in the form of triacylglycerides.

6. A composition according to any one of embodiments 1 or 5, wherein weight ratio between the mono-, di- and triacylglyceride components of the fatty acids in the fatty acid oil mixture is 15-60:25-60:0-45, preferably 15-60:40-60:0-45.

7. A composition according to anyone of embodiments 1 to 6, wherein the fatty acid oil mixture comprises at least 60, preferably at least 70 weight % of at least one fatty acid chosen from EPA and DHA, by weight of the fatty acid oil mixture.

8. A composition according to anyone of embodiments 1 to 7, wherein the fatty acid oil mixture comprises at least 60, preferably at least 65, in particular at least 70, especially at least 75 weight % EPA and DHA, by weight of the fatty acid oil mixture.

9. A composition according to anyone of embodiments 1 to 8, wherein the weight ratio of EPA:DHA is from about 1:10 to about 10:1, preferably from about 1:8 to about 8:1, in particular from about 1:6 to about 6:1, especially from about 1:5 to about 5:1, in particular preferably from about 1:4 to about 4:1.

10. A composition according to anyone of embodiments 1 to 9, wherein the weight ratio of omega-3 fatty acids to omega-6 fatty acids is at least 14:1, preferably at least 16:1, in particular at least 20:1.

11. A composition according to anyone of embodiments 1 to 10 wherein the composition comprises an antioxidant, preferably a tocopherol, such as alpha-tocopherol, beta-tocopherol, gamma-tocopherol or delta-tocopherol, or mixtures thereof; BHA, such as 2-tert-butyl-4-hydroxyanisole or 3-tert-butyl-4-hydroxyanisole, or mixtures thereof; BHT (3,5-di-tert-butyl-4-hydroxytoluene) or ascorbyl palmitate; or mixtures thereof.

12. A composition according to anyone of embodiments 1 to 11, wherein the bioavailability of the fatty acids of the fatty acid oil mixture is at least 20% higher compared to a similar fatty acid ethyl ester composition.

13. A formulation comprising
a composition according to anyone of the embodiments 1 to 12, and
a shell,
wherein the composition is encapsulated in the shell, for use in the therapeutic and/or prophylactic treatment of cachexia.

14. An oral formulation comprising
a composition according to anyone of the embodiments 1 to 12, and
a gelatin capsule,
wherein the composition is encapsulated in the gelatin capsule, preferably in a gelatin capsule containing additives to improve stability and/or taste and smell of the formulation.

15. An oral formulation according to embodiment 14, wherein the gelatine capsule comprises at least gelatin and at least one plasticizer, preferably the at least one plasticizer is glycerol or sorbitol.

16. A tube formulation comprising
a composition according to anyone of the embodiments 1 to 12, and
enteral device,
wherein the composition is filled into the enteral device.

17. Composition according to anyone of the embodiments 1 to 12 for use in the therapeutic and/or prophylactic treatment of cachexia of patients with diagnosed cancer, preferably, with diagnosed lung, gastro, pancreatic and/or head and neck cancer.

18. Composition according to anyone of the embodiments 1 to 12 for use in the therapeutic and/or prophylactic treatment of cachexia, wherein at least one of the following parameter is alleviate or corrected: loss in body mass, loss of muscle strength, weakness/fatigue, loss of appetite, myosteatosis, increase in quality of life and/or reduction of days spent in hospital.

19. Composition according to anyone of embodiments 1 to 12 for use in the treatment of cachexia, wherein the treatment is a nutritional treatment by dietary management of the cachexia.

20. Composition according to anyone of embodiments 1 to 12, for use in the treatment of cachexia, wherein the composition is selected from the group of Enteral Formulas for Special Medical Use, Foods for Specified Health Uses, Food for Special Medical Purposes (FSMP), Food for Special Dietary Use (FSDU), Medical Nutrition and Medical Food, and the use is a nutritional treatment.

21. Formulation according to anyone of the embodiments 13 to 16 for use in the therapeutic and/or prophylactic treatment of cachexia of patients wherein the underlying disease of cachexia is one or more of cancer, chronic heart failure, HIV/AIDS, chronic obstructive pulmonary disease (COPD) and rheumatoid arthritis.

22. Formulation according to anyone of embodiments 13 to 16 for use in the treatment of cachexia, wherein the treatment is a nutritional treatment by dietary management of the cachexia.

23. Formulation according to anyone of embodiments 13 to 16, for use in the treatment of cachexia, wherein the composition is selected from the group of Enteral Formulas for Special Medical Use, Foods for Specified Health Uses, Food for Special Medical Purposes (FSMP), Food for Special Dietary Use (FSDU), Medical Nutrition and Medical Food, and the use is a nutritional treatment.

24. Formulation according to anyone of embodiments 13 to 16 for use in the nutritional management of un-intentional weight loss and/or muscle loss and/or to maintain lean body mass and/or improve quality of life in a cancer patient.

25. A method for preventing and/or treating cachexia in a subject in need thereof, comprising administering to a subject in need a composition according to anyone of embodiment 1 to 12 or a formulation according to anyone of embodiments 13 to 16.

26. The method of embodiment 25, wherein the method comprises administering from about 100 mg to 6 g, preferably from about 200 mg to about 4 g, in particular from about 250 mg to about 3 g, especially from about 300 mg to about 2 g, in particular preferably from about 400 mg to 1 g per day of the composition.

27. The method of embodiment 25 or 26, wherein the method comprises administering from about 200 mg to 4 g, preferably from about 1 to 4 g, in particular from about 1.5 to 3.5 g, especially from about 2 to 3 g per day EPA and DHA.

28. Use of the composition according to anyone of embodiments 1 to 12 or a formulation according to anyone of embodiments 13 to 16 for preventing and/or treating cachexia in a subject in need thereof.

29. Special Medical Use product, Foods for Specified Health Uses product, Food for Special Medical Purposes (FSMP) product, Food for Special Dietary Use (FSDU) product, Medical Nutrition product and Medical Food product comprising a composition according to anyone of embodiments 1 to 12 or a formulation according to anyone of embodiments 13 to 16 for use in the nutritional management of un-intentional weight loss in a human, wherein the treatment improves at least one parameter associated with cachexia, such as to alleviate or correct at least one of: loss in body mass, loss of muscle strength, weakness/fatigue, loss of appetite, myosteatosis, or improves quality of life and/or reduce days spent in hospital for said patient.
